(19) United States
(12) Patent Application Publication
(10) Pub. No.: US 2023/0165980 A1
(43) Pub. Date: Jun. 1, 2023

(54) METHODS FOR RADIOLABELING PSMA BINDING LIGANDS AND THEIR KITS

(71) Applicant: Novartis AG, Basel (CH)

(72) Inventors: Donato BARBATO, Ivrea (IT); Erica MARTINENGO, Torino (IT); Lorenzo SACCHETTI, Torino (IT); Mattia TEDESCO, Alba (Cuneo) (IT)

(21) Appl. No.: 17/997,299

(22) PCT Filed: Apr. 28, 2021
(86) PCT No.: PCT/EP2021/061138

§ 371 Date: Oct. 27, 2022

(30) Foreign Application Priority Data
Apr. 29, 2020 (EP) 20172151.1

(51) Int. Cl. A61K 51/04; A61K 51/12; A61K 47/12

(52) U.S. Cl. CPC A61K 51/0497; A61K 51/121; A61K 47/12

(57) ABSTRACT

The present invention relates to methods for labeling a PSMA binding ligand with a radioactive isotope, preferably ⁶⁸Ga, ⁶⁷Ga or ⁶⁴Cu, said method comprising the steps of: i. providing a single vial comprising, in dried form, said PSMA binding ligand f the following formula (I): (I) at least one buffering agent, sodium chloride and a stabilizer against radiolytic degradation, ii. adding a solution of said radioactive isotope into said single vial, thereby obtaining a solution of said PSMA binding ligand of formula (I) with said radioactive isotope, iii. mixing the solution obtained in ii., and incubating it for a sufficient period of time for obtaining said PSMA binding ligand labelled with said radioactive isotope, and, iv. optionally, adjusting the pH of the solution.

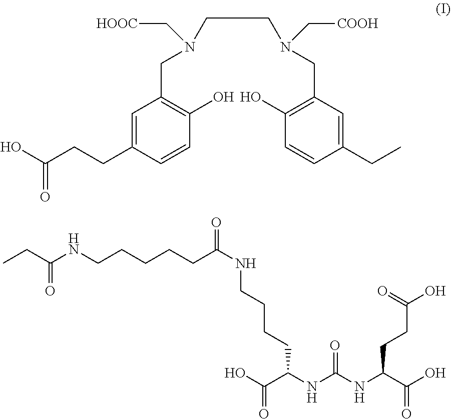

### TECHNICAL FIELD

The present disclosure relates to methods for radiolabeling PSMA binding ligands, and their kits.

### BACKGROUND

Prostate cancer is one of the most widespread cancers in the US and in Europe. In particular, metastatic prostate cancer (mCRPC) is associated with poor prognosis and diminished quality of life.

Recently, a new development stream for treating prostate cancer is represented by the endo-radiotherapy based on PSMA ligands, as PSMA is considered to be a suitable target for imaging and therapy due to its over-expression in primary cancer lesions and in soft-tissue/bone metastatic disease. Also, PSMA expression seems to be even higher in the most aggressive castration-resistant variants of the disease, which represents a patient population with high unmet medical need. (Marchal et al., Histol Histopathol, 2004, July; 19(3):715-8; Mease et al., Curr Top Med Chem, 2013, 13(8):951-62).

Among many small-molecule ligands targeting PSMA, the urea-based low molecular weight agents have been the most extensively investigated ones. These agents were shown to be suitable for prostate cancer clinical assessment as well as for PRRT therapy (Kiess et al., Q J Nucl Med Mol Imaging, 2015; 59:241-68). Some of these agents have glutamate-urea-lysine (GUL) as the targeting scaffold. A class of molecules was created following the strategy to attach a linker between the chelator and GUL moiety. This approach allows the urea to reach the binding site while keeping the metal chelated portion on the exterior of the binding site. This strategy was successful in xenograft PSMA positive tumors due to its demonstrated high uptake and retention as well as fast renal clearance (Banerjee et al., J Med Chem, 2013; 56:6108-21). It has also been shown that this class of molecule can be labelled with ⁶⁸Ga, and used it in the detection of prostate cancer lesions by PET imaging (Eder et al. Pharmaceuticals 2014, 7, 779-796).

Patent application US2016/0256579A1 report a PSMA binding agent kit. However, no optimized method has been developed for labeling PSMA binding ligand with ⁶⁸Ga, ⁶⁷Ga or ⁶⁴Cu to thereby obtain labelled PSMA binding ligand solution for imaging purposes of prostate cancer tumors in human patients. In particular, there is need for a rapid, efficient, robust and safe procedure which would provide a high radiochemical purity of labelled PSMA binding ligand, such as [⁶⁸Ga] PSMA binding ligand for intravenous injection in human subject in need thereof.

### SUMMARY

One first aspect of the disclosure relates to a method for labeling a PSMA binding ligand with a radioactive isotope, preferably ⁶⁸Ga, ⁶⁷Ga or ⁶⁴Cu, said method comprising the steps of:
i. providing a single vial comprising, in dried form, said PSMA binding ligand of the following formula (I):

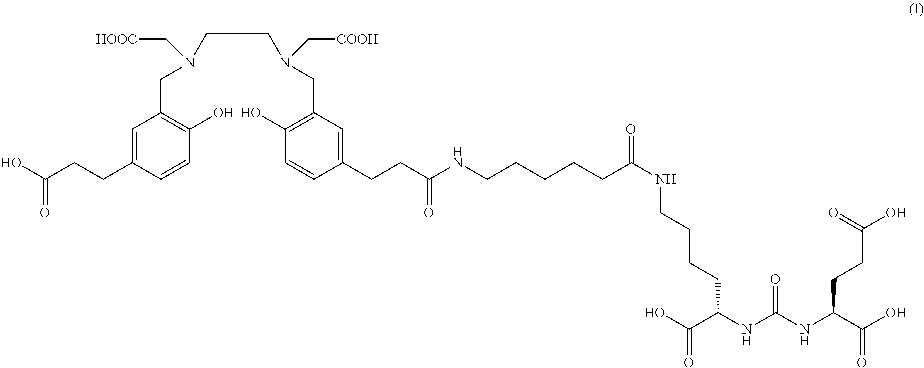 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

at least one buffering agent, sodium chloride and a stabilizer against radiolytic degradation,
ii. adding a solution of said radioactive isotope into said single vial, thereby obtaining a solution of said PSMA binding ligand of formula (I) with said radioactive isotope,
iii. mixing the solution obtained in ii., and incubating it for a sufficient period of time for obtaining said PSMA binding ligand labelled with said radioactive isotope, and,
iv. optionally, adjusting the pH of the solution.

In another aspect, the disclosure relates to a solution comprising a PSMA binding ligand of formula (I) labelled with a radioactive isotope, obtainable or obtained by the method, for use as an injectable solution for in vivo detection of tumors, preferably PSMA-expressing tumors, by imaging in a subject in need thereof

It is another object of the present disclosure to provide a powder for solution for injection, comprising the following components in dried forms:
i. a PSMA binding ligand of formula (I):

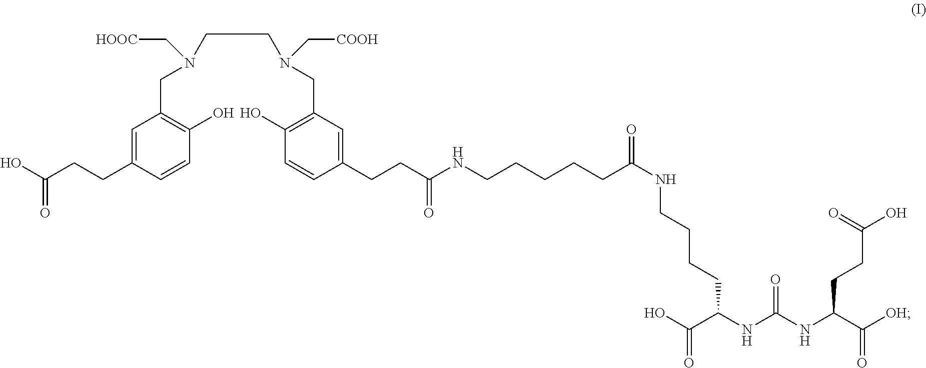 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

ii. sodium chloride;
iii. at least one buffering agents preferably, sodium acetate and;
iv. a stabilizer against radiolytic degradation, preferably gentisic acid.

Preferably, said powder for solution for injection comprises the following components:
i. a PSMA binding ligand of formula (I) in an amount between 5 μg and 60 μg, preferably between 10 and 40 μg, more preferably between 15 and 30 μg, even more preferably about 25 μg;

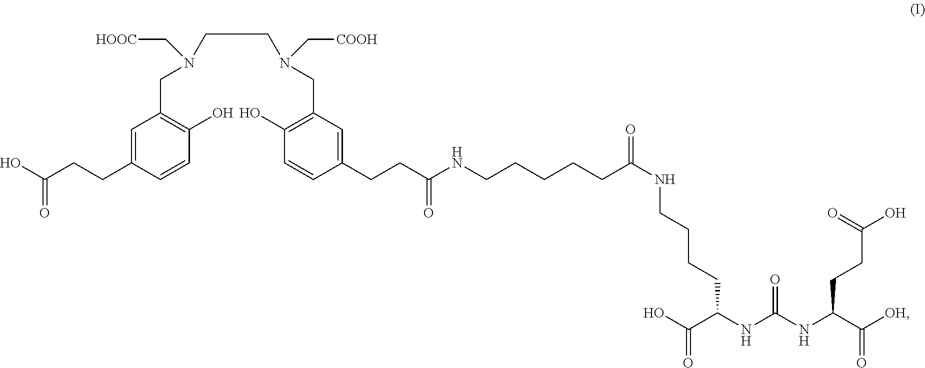 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg and;
iv. gentisic acid in an amount of at least 0.2 mg, preferably between 0.5 mg and 2 mg, more preferably between 0.8 mg and 1.2 mg, even more preferably about 1 mg.

The present disclosure further relates to a kit for carrying out the method, comprising
i. a single vial with the following components in dried forms
   i. a PSMA binding ligand of formula (I):

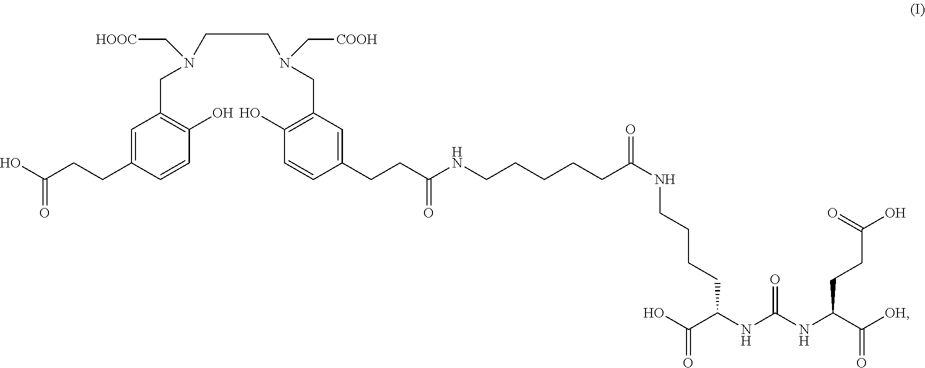 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride;
iii. at least one buffering agent, preferably sodium acetate;
iv. a stabilizer against radiolytic degradation, preferably gentisic acid;
v. optionally, an accessory cartridge for eluting a radioactive isotope generated by a radioactive isotope generator or cyclotron.

Another kit herein disclosed comprises
i. a single vial with the following components, preferably in dried forms:
   i. a PSMA binding ligand of formula (I):

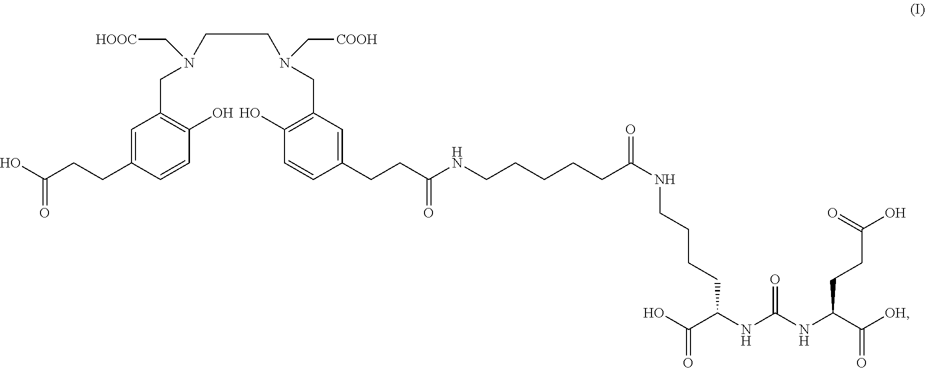 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride;
iii. sodium acetate;
iv. a stabilizer against radiolytic degradation, preferably gentisic acid, and;
v. optionally, an accessory cartridge for eluting a radioactive isotope generated by a radioactive isotope generator or cyclotron.

Preferably, the kit may comprise a single vial with the following components:
i. a PSMA binding ligand of formula (I) in an amount between 5 μg and 60 μg, preferably between 10 μg and 40 μg, more preferably between 15 μg and 30 μg, even more preferably about 25 μg;

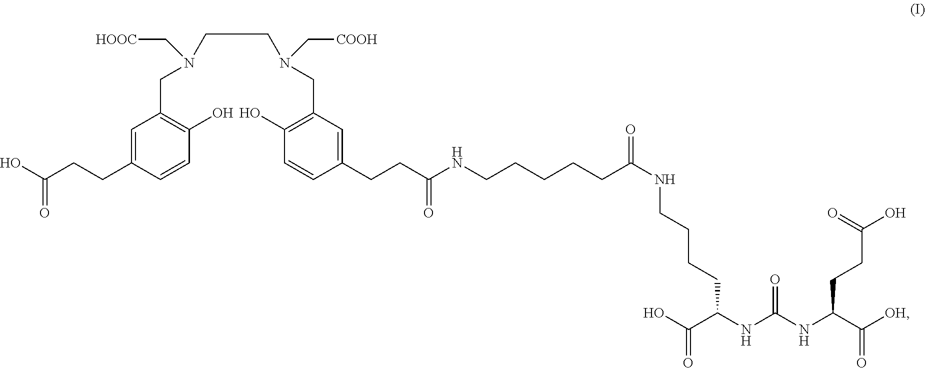 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg;
iv. gentisic acid in an amount at least 0.2 mg, preferably between 0.5 mg and 2 mg, more preferably between 0.8 mg and 1.2 mg, even more preferably about 1 mg, and;
v. optionally, an accessory cartridge for eluting a radioactive isotope generated by a radioactive isotope generator or cyclotron.

### DETAILED DESCRIPTION OF THE INVENTION

In general, the present disclosure relates to a method for labeling a PSMA binding ligand with a radioactive isotope, preferably ⁶⁸Ga, ⁶⁷Ga or ⁶⁴Cu, said method comprising the steps of:
i. providing a single vial comprising, in dried form, said PSMA binding ligand of the following formula (I):

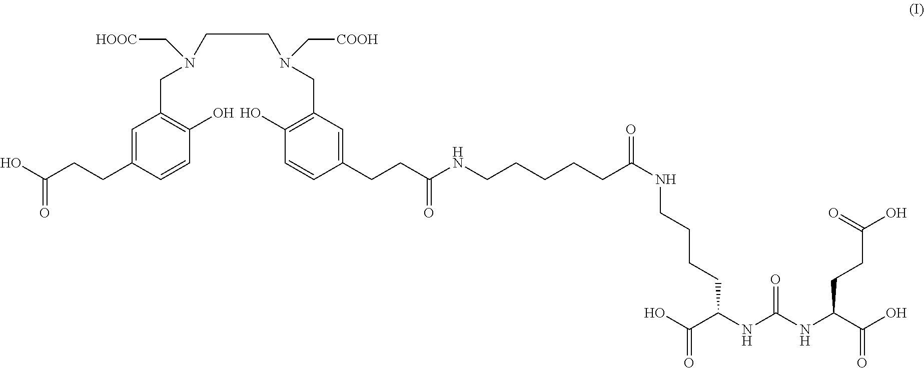 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

at least one buffering agent, sodium chloride and a stabilizer against radiolytic degradation,
ii. adding a solution of said radioactive isotope into said single vial, thereby obtaining a solution of said PSMA binding ligand of formula (I) with said radioactive isotope,
iii. mixing the solution obtained in ii., and incubating it for a sufficient period of time for obtaining said PSMA binding ligand labelled with said radioactive isotope, and,
iv. optionally, adjusting the pH of the solution.

The radiolabelled PSMA binding ligand obtained by the disclosed methods is preferably a radioactive PSMA binding ligand for use as a contrast agent for PET/CT, SPECT or

PET/MRI imaging. In a preferred embodiment, ⁶⁷Ga is used for SPECT imaging and ⁶⁸Ga and ⁶⁴Cu are used for PET imaging such as PET/CT or PET/MRI imaging.

The radiolabelled PSMA binding ligand obtained by the disclosed methods is the PSMA binding ligand of formula (I):

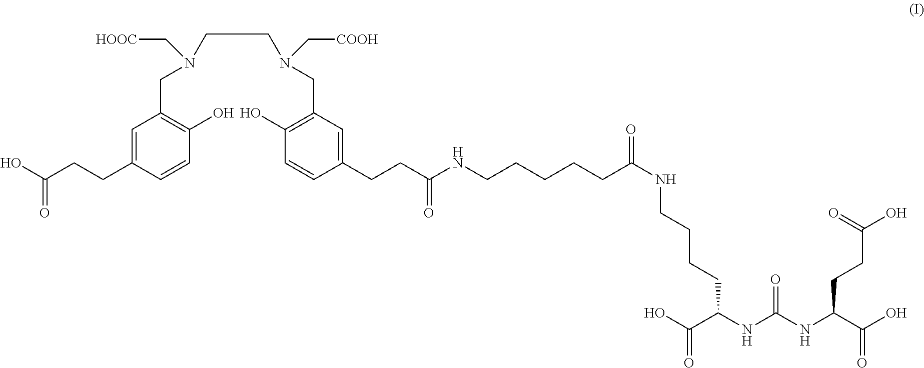 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

labelled with a radioactive isotope suitable for use as a contrast agent for PET/CT, SPECT or PET/MRI imaging, preferably ⁶⁸Ga, ⁶⁷Ga or ⁶⁴Cu.

The methods of the present disclosure may advantageously provide excellent radiochemical purity of the radiolabelled compound, e.g. radiolabelled PSMA binding ligand of formula (I) with ⁶⁸Ga, ⁶⁷Ga or ⁶⁴Cu, preferably the radiochemical purity as measured in HPLC is at least 91%, and optionally, the percentage of free ⁶⁸Ga3+, ⁶⁷Ga3+ or ⁶⁴Cu²⁺ (in HPLC) is 3% or less, and/or the percentage of not complexed ⁶⁸Ga3+, ⁶⁷Ga3+ or ⁶⁴Cu²⁺ species (in ITLC) is 3% or less. Assays for measuring radiochemical purity in HPLC or in ITLC and free ⁶⁸Ga3+ are further described in detail in the Examples.

### Definitions

The terms “PSMA binding ligand” and “PSMA ligand” are used interchangeably in the present disclosure. They refer to a molecule capable of interacting, preferably binding, with the PSMA enzyme.

The phrase “treatment of” and “treating” includes the amelioration or cessation of a disease, disorder, or a symptom thereof. In particular, with reference to the treatment of a tumor, the term “treatment” may refer to the inhibition of the growth of the tumor, or the reduction of the size of the tumor.

Consistent with the International System of Units, “MBq” is the abbreviation for the unit of radioactivity “megabecquerel.”

As used herein, “PET” stands for positron-emission tomography.

As used herein, “SPECT” stands for single-photon emission computed tomography.

As used herein, “MM” stands for magnetic resonance imaging.

As used herein, “CT” stands for computed tomography.

As used herein, the terms “effective amount” or “therapeutically efficient amount” of a compound refer to an amount of the compound that will elicit the biological or medical response of a subject, preferably ameliorate the symptoms, alleviate conditions, slow or delay disease progression, or prevent a disease.

As used herein, the term “in dried form” refers to a pharmaceutical composition that has been dried to a powder having a moisture content below about 10% by weight, usually below about 5% by weight, and preferably below about 3%.

As used herein, the term “chelator” refers to a molecule with functional groups such as amines or carboxylic group suitable to complex the radioactive isotope via non-covalent bonds.

As used herein, the term “stabilizer against radiolytic degradation” refers to a stabilizing agent which protects organic molecules against radiolytic degradation, e.g. when a gamma ray emitted from the radionuclide is cleaving a bond between the atoms of an organic molecules and radicals are formed, those radicals are then scavenged by the stabilizer which avoids the radicals undergoing any other chemical reactions which might lead to undesired, potentially ineffective or even toxic molecules. Therefore, those stabilizers are also referred to as “free radical scavengers” or in short “radical scavengers”. Other alternative terms for those stabilizers are “radiation stability enhancers”, “radiolytic stabilizers”, or simply “quenchers”. Such as para-aminobenzoic acid (PABA), Ascorbic Acid, Gentisic Acid, Sodium Metabisulfite, Aminobenzoic acid, Lipoic Acid.

As used herein, the term “Radiochemical purity” refers to that percentage of the stated radionuclide that is present in the stated chemical or biological form. Radiochromatography methods, such as HPLC method or instant Thin Layer Chromatography method (iTLC), are the most commonly accepted methods for determining radiochemical purity in the nuclear pharmacy.

If not stated herein otherwise, “about” means ±20%, preferably ±10%, more preferably ±5%, even more preferably ±2%, even more preferably ±1%. The term “about” is herein used synonymous with “ca.”

As used herein, when referring to the weight of sodium acetate, it is meant the weight of the anhydrous salt of sodium acetate.

Step (i) of Providing a Single Vial Comprising Said PSMA Binding Ligand in Dried Form

The PSMA Binding Ligand

Examples of said PSMA binding ligands are disclosed in US2015/110715 or in Clemens Kratochwil et al. “PSMA-Targeted Radionuclide Therapy of Metastatic Castration-Resistant Prostate Cancer with ¹⁷⁷Lu-Labeled PSMA-617”, THE JOURNAL OF NUCLEAR MEDICINE, Vol. 57, No. 8, August 2016.

Advantageously, the PSMA binding ligand is a molecule comprising a) a urea of 2 amino-acid residues, preferably a glutamate-urea-lysine (GUL) moiety, and b) a chelating agent which can coordinate radioactive isotope.

In specific embodiments, the PSMA binding ligand is a compound of formula (I):

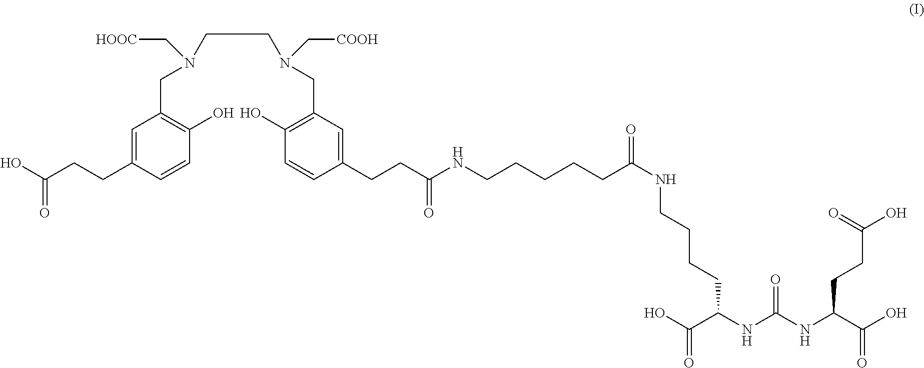 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

The Single Vial Comprising Said PSMA Binding Ligand

In the present invention the radiolabeling method uses a single vial kit. In this embodiment, said single vial comprises said PSMA binding ligand, at least one buffering agent, sodium chloride and a stabilizer against radiolytic degradation, all in dried forms.

Preferably, said PSMA binding ligand, more preferably PSMA binding ligand of formula (I), is comprised in said single vial in an amount between 5 μg and 60 μg, preferably between 10 μg and 40 μg, more preferably between 15 μg and 30 μg, even more preferably about 25 μg.

In preferred embodiments, said at least one buffering agent is sodium acetate. Preferably, said sodium acetate is present in an amount of at least 20 mg, preferably between 20 mg and 80 mg, more preferably between 42 mg and 52 mg, even more preferably about 47 mg.

Preferably, said sodium chloride is present in an amount of at least 10 mg, preferably between 10 mg and 100 mg, more preferably between 30 mg and 50 mg, even more preferably about 40 mg.

In preferred embodiments, said stabilizer against radiolytic degradation is gentisic acid. Preferably, said gentisic acid is present in an amount of at least 0.2 mg, preferably between 0.5 mg and 2 mg, more preferably between 0.8 mg and 1.2 mg, even more preferably 1 mg. Preferably, the stabilizer against radiolytic degradation consists essentially of gentisic acid. Preferably, the single vial kit does not comprise ascorbic acid or ethanol.

In specific embodiment, the single vial does not contain any bulking agent selected from the group consisting of carbohydrate (e.g. mono- or di- or poly-saccharides) and polymeric agent. Preferably, the single vial does not contain any of the following bulking agents: mannitol, maltose, trehalose, polyvinylpyrrolidone and mixtures thereof.

A preferred example of said single vial is given in the examples.

The single vial is preferably obtained by freeze-drying using methods well known in the art. Therefore, said single vial may be provided in a lyophilized or spray dried form.

As used herein, the buffering agent is a buffer suitable for obtaining a pH from 3.0 to 6.0, at the incubating step (iii). A “buffer for a pH from 3.0 to 6.0” may advantageously be a sodium acetate buffer.

In a specific embodiment, the single vial does not contain any bulking agent selected from the group consisting of carbohydrate (e.g. mono- or di- or poly-saccharides) and polymeric agent. In particular, the single vial does not contain any of the following bulking agents: mannitol, maltose, trehalose, polyvinylpyrrolidone and mixtures thereof, and the buffering agent is a buffer suitable for obtaining a pH from 3.0 to 6.0, at the incubating step (iii).

Step (ii) of Adding a Solution of Said Radioactive Isotope into Said Single Vial

Radioactive isotopes for use in the radiolabeling methods include those suitable as contrast agent in PET and SPECT imaging, preferably selected from the group consisting of:

¹¹¹In, ^{133m}In, ^{99m}Tc, ^{94m}Tc, ⁶⁷Ga, ⁶⁶Ga, ⁶⁸Ga, ⁵²Fe, ⁷²As, ⁹⁷Ru, ²⁰³Pb, ⁶²Cu, ⁶⁴Cu, ⁸⁶Y, ^{51q}Cr, ^{52m}Mn, ¹⁵⁷Gd, ¹⁶⁹Yb, ¹⁷²Tm, ^{177m}Sn, ⁸⁹Zr, ⁴³Sc, ⁴⁴Sc, ⁵⁵Co.

According to a preferred embodiment, the radioactive isotope is ⁶⁸Ga, ⁶⁷Ga or ⁶⁴Cu. In a preferred embodiment, ⁶⁷Ga is used for SPECT imaging and ⁶⁸Ga and ⁶⁴Cu are used for PET imaging such as PET/CT or PET/MRI imaging.

The metallic ions of such radioisotopes are able to form non-covalent bond with the functional groups of the chelator, e.g. carboxylic acids of the HBED-CC chelating agent.

In a specific embodiment, said solution of said radioactive isotope is an eluate obtained from the steps of
i. producing a radioactive isotope from a parent non-radioactive element by means of a radioactive isotope generator or a cyclotron,
ii. separating said radioactive isotope from said parent non-radioactive element by elution in HCl as an elution solvent, and
iii. recovering the eluate,
   thereby obtaining a solution of said radioactive isotope in HCl.

In specific embodiments, the solution containing said radioactive isotope is an aqueous solution comprising the radioisotope is in the form of a metal ion, e.g. ⁶⁸Ga³⁺, ⁶⁷Ga³⁺ or ⁶⁴Cu²⁺. The solution containing said radioactive isotope can be an aqueous solution comprising ⁶⁸GaCl₃, ⁶⁷GaCl₃ or ⁶⁴CuCl₂, in HCl.

Said solution comprising the radioactive isotope ⁶⁸Ga may be an eluate preferably obtained from the steps of:
i. producing ⁶⁸Ga from the parent ⁶⁸Ge by means of a generator, and
ii. optionally, separating the generated ⁶⁸Ga from ⁶⁸Ge by passing ⁶⁸Ge/⁶⁸Ga through a suitable cartridge, and eluting ⁶⁸Ga in HCl,
   thereby obtaining a solution of said radioactive isotope in HCl.

Such methods of producing ⁶⁸Ga from ⁶⁸Ge/⁶⁸Ga generators are well-known in the art and for example described in Martiniova L. et al. “Gallium-68 in Medical Imaging”, Curr Radiopharm., 2016, 9(3), pp 187-20; Dash A, Chakravarty, “Radionuclide generators: the prospect of availing PET radiotracers to meet current clinical needs and future research demands”, R. Am. J. Nucl. Med. Mol. Imaging., 2019 Feb. 15, 9(1), pp. 30-66.

Said solution comprising the radioactive isotope ⁶⁸Ga may be an eluate preferably obtained from cyclotron production. Such production is for example described in Am J Nucl Med Mol Imaging 2014; 4(4):303-310 or in B. J. B. Nelson et al./Nuclear Medicine and Biology 80-81, (2020), pp. 24-31.

Preferably, ⁶⁸Ga may be produced by a cyclotron, more preferably using a proton beam of energy between 8 and 18 MeV, even more preferably between 11 and 14 MeV. The ⁶⁸Ga may be produced via the ⁶⁸Zn(p,n)⁶⁸Ga reaction using a solid or liquid target system. The target consists of enriched ⁶⁸Zn metal or ⁶⁸Zn liquid solution. After irradiation, the target is transferred for further chemical processing in which the ⁶⁸Ga is isolated using ion exchange chromatography. ⁶⁸Ga is eluted in HCl solution.

Alternatively, said radioactive isotope is ⁶⁷Ga. Various methods for the production of ⁶⁷Ga, using either a zinc (enriched or natural) or copper or germanium target with protons, deuterons, alpha particles or helium(III) as the bombarding particle, have been reported as summarised by Helus, F., Maier-Borst, W., 1973. A comparative investigation of methods used to produce 67Ga with a cyclotron. In: Radiopharmaceuticals and Labelled Compounds, Vol. 1, IAEA, Vienna, pp. 317-324, M. L Thakur Gallium-67 and indium-111 radiopharmaceuticals Int. J. Appl. Rad. Isot., 28 (1977), pp. 183-201, and Bjørnstad, T., Holtebekk, T., 1993. Production of ⁶⁷Ga at Oslo cyclotron. University of Oslo Report OUP8-3-1, pp. 3-5. Bombardment of ^{nat}Ge targets with moderate energy protons (up to 64 MeV) is also a suitable method to produce 67Ga as described in T Horiguchi, H Kumahora, H Inoue, Y Yoshizawa Excitation functions of Ge(p,xnyp) reactions and production of 68Ge, Int. J. Appl. Radiat. Isot., 34 (1983), pp. 1531-1535.

Preferably, ⁶⁷Ga may be produced by a cyclotron. Such methods of producing ⁶⁷Ga from ⁶⁸Zn (p, 2n)⁶⁷Ga are well-known in the art and for example described in Alirezapour B et al. Iranian Journal of Pharmaceutical Research (2013), 12 (2): 355-366. More preferably, this method uses a proton beam of energy between 10 and 40 MeV. The ⁶⁷Ga may be produced via either the ⁶⁷Zn (p, n)⁶⁷Ga or either the ⁶⁸Zn (p, 2n)⁶⁷Ga reaction using a solid or liquid target system. The target consisted of enriched ⁶⁷Zn or ⁶⁸Zn metal or liquid solution. After irradiation, the target is transferred for further chemical processing in which the ⁶⁷Ga is isolated using ion exchange chromatography. Final evaporation from aq. HCl yield ⁶⁷GaCl₃, which may then be added to said single vial for the labeling method.

Alternatively, said radioactive isotope is ⁶⁴Cu as obtained from cyclotron production. Such production method is for example described in WO2013/029616.

Preferably, ⁶⁴Cu may be produced by a cyclotron, more preferably using a proton beam of energy between 11 and 18 MeV. The ⁶⁴Cu may be produced via the ⁶⁴Ni (p,n)⁶⁴Cu reaction using a solid or liquid target system. The target consisted of ⁶⁴Ni metal or ⁶⁴Ni liquid solution. After irradiation, the target is transferred for further chemical processing in which the ⁶⁴Cu is isolated using ion exchange chromatography. Final evaporation from aq. HCl yield ⁶⁴CuCl₂, which may then be added to said single vial for the labeling method.

Step (iii) of Mixing the Solution Obtained in Step (ii) and Incubating it for a Sufficient Period of Time for Obtaining Said PSMA Binding Ligand Labelled with Said Radioactive Isotope

The radiolabeling starts after the mixing of the single vial comprising the PSMA binding ligand (e.g. the PSMA binding ligand of formula (II)) with the solution comprising the radioactive isotope (preferably, ⁶⁸Ga, ⁶⁷Ga or ⁶⁴Cu as disclosed above) in a suitable buffering agent as disclosed above.

In an embodiment, the incubating step is performed at a temperature between 50° C. to 100° C. In specific embodiments, the incubating step is performed for a period of time comprised between 2 and 25 minutes.

In specific embodiments, the incubating step is performed at a temperature between 80° C. and 100° C., preferably between 90° C. and 100° C., more preferably at about 95° C.

In other specific embodiments, the incubating step is performed at a temperature between 50° C. and 90° C., preferably between 60° C. and 80° C., typically at about 70° C.

In other specific embodiments, the incubating step is performed at a temperature between room temperature and 80° C., preferably between 18° C. and 25° C., more preferably at room temperature.

In specific embodiments, the incubating step is performed for a period of time comprised between 2 and 20 minutes, preferably between 3 and 8 minutes, more preferably about 5 minutes.

In other specific embodiments, the incubating step is performed for a period of time comprised between 5 and 25 minutes, preferably between 10 and 20 minutes, more preferably between 12 and 18 minutes, even more preferably about 15 minutes.

In other specific embodiments, the incubating step is performed for a period of time comprised between 10 and 120 minutes preferably between 30 and 60 minutes.

At the end of labeling process, a sequestering agent having a particular affinity for the radioactive isotope (such as ⁶⁸Ga, ⁶⁷Ga or ⁶⁴Cu) may be added to chelate the non-reacted part of the isotope. This complex formed by the sequestering agent and the non-reacted radioactive isotope may then be discarded to increase the radiochemical purity after radiolabeling.

Preferred Embodiments of the Methods for Radiolabeling the PSMA Binding Ligand of Formula (I) with ⁶⁸Ga

The present disclosure more particularly relates to a method for labeling a PSMA binding ligand of formula (I) with ⁶⁸Ga, comprising the steps of:
i. providing a single vial comprising, in dried form, said PSMA binding ligand of the following formula (I)

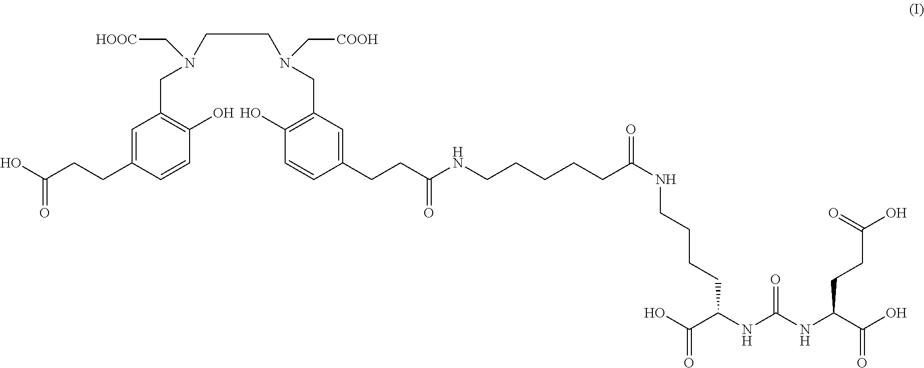 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

at least one buffering agent, sodium chloride and a stabilizer against radiolytic degradation,
ii. adding a solution of said radioactive isotope into said single vial, thereby obtaining a solution of said PSMA binding ligand of formula (I) with said radioactive isotope,
iii. mixing the solution obtained in ii., and incubating it for a sufficient period of time for obtaining said PSMA binding ligand labelled with said radioactive isotope, and,
iv. optionally, adjusting the pH of the solution.

In specific embodiments of said methods, said solution of said ⁶⁸Ga in HCl is an eluate obtained from the steps of
i. producing ⁶⁸Ga element from a parent element ⁶⁸Ge, by means of a generator, and
ii. optionally, separating the generated ⁶⁸Ga element from ⁶⁸Ge element by passing the elements ⁶⁸Ga/⁶⁸Ge through a suitable cartridge, and eluting ⁶⁸Ga in HCl,
   thereby obtaining a solution of said radioactive isotope in HCl.

Preferably, said buffering agent consist of at least 20 mg, preferably between 20 mg and 80 mg, more preferably between 42 mg and 52 mg, even more preferably about 47 mg of sodium acetate.

Preferably, said sodium chloride consist of at least 10 mg, preferably between 10 mg and 100 mg, more preferably between 30 mg and 50 mg, even more preferably about 40 mg.

Preferably said stabilizer against radiolytic degradation consists of 0.2 mg, preferably between 0.5 mg and 2 mg, more preferably between 0.8 mg and 1.2 mg, even more preferably about 1 mg of gentisic acid.

Advantageously, in specific embodiments, a simple labeling of the PSMA binding ligand may be obtained with an eluate of ⁶⁸Ga in HCl coming from commercially available ⁶⁸Ge/⁶⁸Ga generators without any processing of the eluate or any additional purification step.

Powder for a Solution for Injection

The disclosure further relates to a powder for solution for injection, comprising the following components in dried forms:
i. a PSMA binding ligand of formula (I):

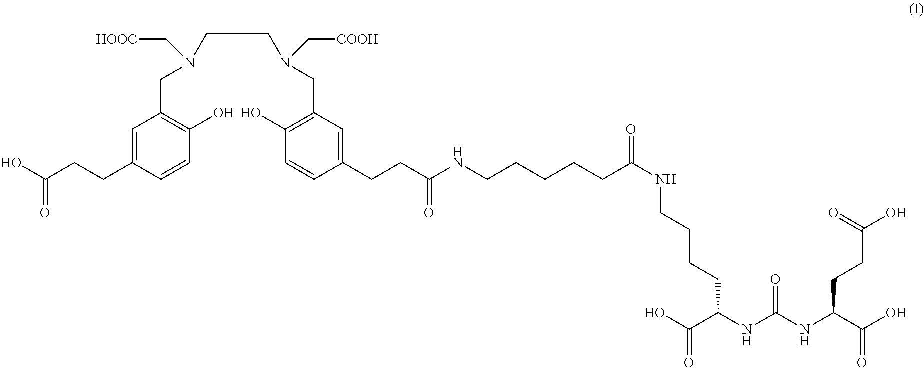 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

ii. sodium chloride;
iii. at least one buffering agent, preferably sodium acetate and;
iv. a stabilizer against radiolytic degradation, preferably gentisic acid.

A preferred embodiment comprises the following components:
i. a PSMA binding ligand of formula (I) in an amount between 5 μg and 60 μg, preferably between 10 μg and 40 μg, more preferably between 15 μg and 30 μg, even more preferably about 25 μg;

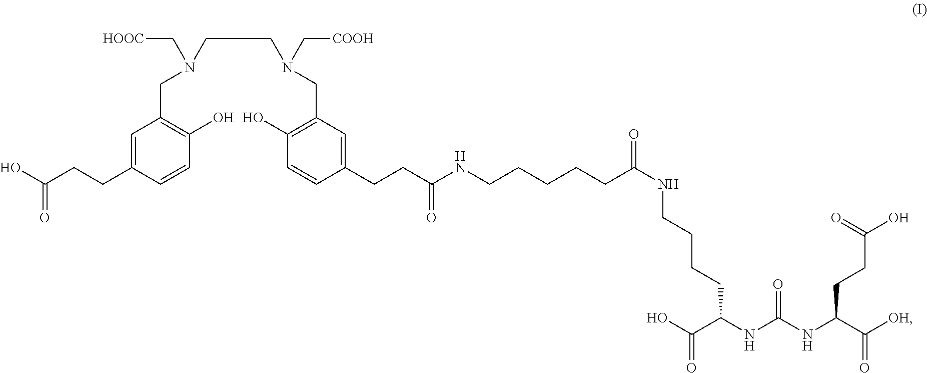 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg and;
iv. gentisic acid in an amount of at least 0.2 mg, preferably between 0.5 mg and 2 mg, more preferably between 0.8 mg and 1.2 mg, even more preferably about 1 mg.

In a specific embodiment, the powder for a solution for injection does not contain any bulking agent selected from the group consisting of carbohydrate (e.g. mono- or di- or poly-saccharides) and polymeric agent. Preferably, the single vial does not contain any of the following bulking agents: mannitol, maltose, trehalose, polyvinylpyrrolidone and mixtures thereof. In a specific embodiment, the stabilizer against radiolytic degradation consists essentially of gentisic acid. Preferably, the powder for a solution for injection does not comprise ascorbic acid or ethanol.

Radiolabeling Kits of the Disclosure

The present disclosure also relates to a kit for carrying out the above labeling methods, said kit comprising
i. a single vial with the following components in dried forms
   i. a PSMA binding ligand of formula (I):

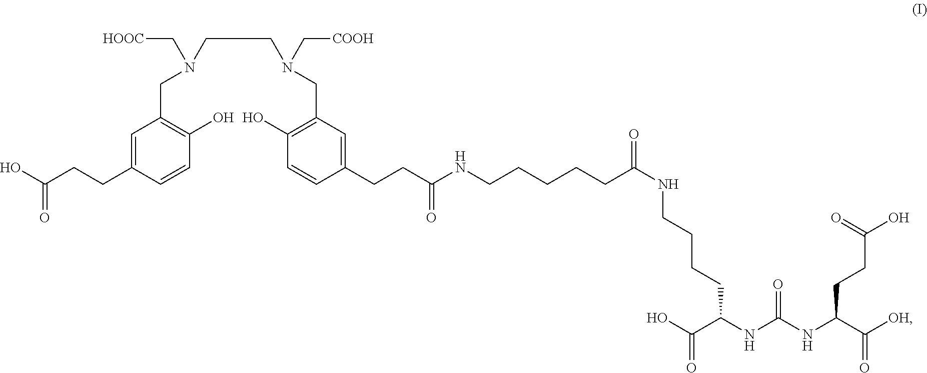 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride;
iii. at least one buffering agent, preferably sodium acetate;
iv. a stabilizer against radiolytic degradation, preferably gentisic acid;
v. optionally, an accessory cartridge for eluting a radioactive isotope generated by a radioactive isotope generator or cyclotron.

In preferred embodiments, said at least one buffering agent is sodium acetate.

In preferred embodiments, said stabilizer against radiolytic degradation is gentisic acid. In preferred embodiments, said stabilizer against radiolytic degradation consists essentially of gentisic acid.

Preferably, said single vial comprises the following components:
i. a PSMA binding ligand of formula (I) in an amount between 5 μg and 60 μg, preferably between 10 μg and 40 μg, more preferably between 15 μg and 30 μg, even more preferably about 25 μg;

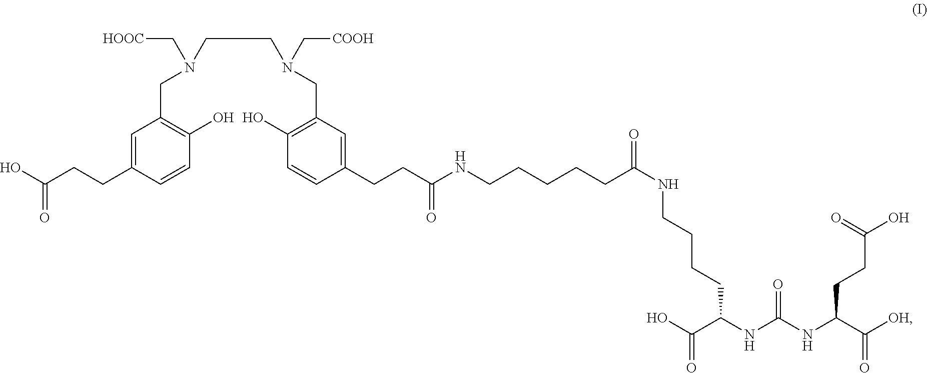 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg;
iv. gentisic acid in an amount of at least 0.2 mg, preferably between 0.5 mg and 2 mg, more preferably between 0.8 mg and 1.2 mg, even more preferably about 1 mg, and;
v. optionally, an accessory cartridge for eluting a radioactive isotope generated by a radioactive isotope generator or cyclotron.

Said single vial may comprise buffering agents for maintaining a pH between 3.0 and 6.0. Preferably, said single vial comprises sodium acetate as buffering agent.

In a specific embodiment, the single vial does not contain any bulking agent selected from the group consisting of carbohydrate (e.g. mono- or di- or poly-saccharides) and polymeric agent. Preferably, the single vial does not contain any of the following bulking agents: mannitol, maltose, trehalose, polyvinylpyrrolidone and mixtures thereof.

Preferably, the single vial, the first or second vial do not comprise ascorbic acid or ethanol.

Preferably, all components of said first, second or single vial are in dried forms.

The radioactive isotope for labeling the PSMA binding ligand may be provided with the kit as ready-for-use product, i.e. for mixing and incubating with the single vial as provided by the kit, or alternatively may be eluted from a radioactive isotope generator prior to, and shortly before mixing and incubating with said single vial, particularly in cases said radioactive isotope has a relatively short half-life such as ⁶⁸Ga, ⁶⁷Ga and ⁶⁴Cu.

Preferably, the components are inserted into sealed containers which may be packaged together, with instructions for performing the method according to the present disclosure.

The kit may be applied in particular for use in the methods as disclosed in the next section.

In a specific embodiment, the kit does not contain any bulking agent selected from the group consisting of carbohydrate (e.g. mono- or di- or poly-saccharides) and polymeric agent. Preferably, the kit does not contain any of the following bulking agents: mannitol, maltose, trehalose, polyvinylpyrrolidone and mixtures thereof.

In a specific embodiment, the kit does not contain any bulking agent selected from the group consisting of carbohydrate (e.g. mono- or di- or poly-saccharides) and polymeric agent. Preferably the kit does not contain any of the following bulking agents: mannitol, maltose, trehalose, polyvinylpyrrolidone and mixtures thereof and said single vial comprises buffering agents for maintaining a pH between 3.0 and 6.0.

In specific embodiments, the PSMA binding ligand is the PSMA binding ligand of formula (I) as defined above.

Use of the Kit According to the Present Disclosure

The above-defined kits may be applied in particular for use of the labeling methods as disclosed in the previous sections.

Advantageously, a solution comprising a PSMA binding ligand (e.g. PSMA binding ligand of formula (I)) labelled with a radioactive isotope (for example ⁶⁸Ga, ⁶⁷Ga or ⁶⁴Cu) is obtainable or obtained by the labeling methods as disclosed in the previous sections.

Such solution may be ready for use as an injectable solution, preferably for in vivo detection of tumors by imaging in a subject in need thereof.

In certain aspects the subject is a mammal, for example but not limited to a rodent, canine, feline, or primate. In preferred aspects, the subject is a human.

The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and SHP Handbook on Injectable Drugs, Trissel, 15th ed., pages 622-630 (2009)).

Preferably, said solution for use as an injectable solution provides a single dose between 1.0 and 3.0 MBq more preferably between 1.8 and 2.2 MBq per kilogram bodyweight of [⁶⁸Ga]-PSMA binding ligand of formula (I) for administration to a subject in need thereof.

In specific embodiments, said subject in need thereof a subject has a cancer having PSMA expressing tumor or cells. The PSMA-expressing tumor or cell can be selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an oesophageal tumor or cell, a stomach tumor or cell, and combinations thereof. In some other embodiments, the PSMA-expressing tumors or cells is a prostate tumor or cell

Preferably, PET/MRI, SPECT or PET/CT imaging may be acquired 20 to 120 minutes, more preferably between 50 to 100 minutes, after the intravenous administration of the radiolabelled PSMA binding ligand to the subject, and even more preferably about 1 hour after the administration of the radiolabelled PSMA binding ligand to the subject. The minimum recommended time to wait before starting PET/MRI, SPECT or PET/CT imaging is 50 minutes after the intravenous administration.

Synthesis of the Compounds of Formula (I)

The compounds of formula (I) can be synthesized using the methods disclosed in Matthias Eder, Martin Schafer, Ulrike Bauder-Wust, William-Edmund Hull, Carmen Wangler, Walter Mier, Uwe Haberkorn, and Michael Eisenhut “68Ga-Complex Lipophilicity and the Targeting Property of a Urea-Based PSMA Inhibitor for PET Imaging”—Bioconjugate Chem. 2012, 23, 688-697 or is commercially available via ABX advanced biochemical compounds.

### Embodiments

The following specific embodiments are disclosed:
1. A method for labeling a PSMA binding ligand with a radioactive isotope, preferably ⁶⁸Ga, ⁶⁷Ga or ⁶⁴Cu, said method comprising the steps of:
   i. providing a single vial comprising, in dried form, said PSMA binding ligand of the following formula (I):

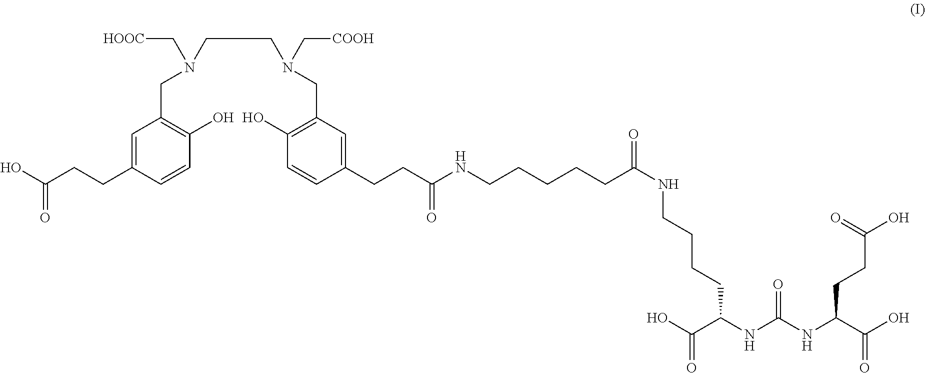 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

at least one buffering agent, sodium chloride and a stabilizer against radiolytic degradation,
ii. adding a solution of said radioactive isotope into said single vial, thereby obtaining a solution of said PSMA binding ligand of formula (I) with said radioactive isotope,
iii. mixing the solution obtained in ii., and incubating it for a sufficient period of time for obtaining said PSMA binding ligand labelled with said radioactive isotope, and,
iv. optionally, adjusting the pH of the solution.
2. The method of Embodiment 1, wherein said at least one buffering agent is sodium acetate.
3. The method of any one of Embodiment 1 or 2, wherein said single vial does not contain any bulking agent selected from the group consisting of carbohydrate and polymeric agent, preferably the single vial does not contain any of the following bulking agents: mannitol, maltose, trehalose, polyvinylpyrrolidone and mixtures thereof, and the buffering agent is a buffer suitable for obtaining a pH from 3.0 to 6.0, at the incubating step (iii).
4. The method of any one of Embodiments 1-3, wherein said PSMA binding ligand of formula (I) is comprised in said single vial in an amount between 5 μg and 60 μg, preferably between 10 μg and 40 μg, more preferably between 15 μg and 30 μg, even more preferably about 25 μg.
5. A solution comprising a PSMA binding ligand of formula (I) labelled with a radioactive isotope, obtainable or obtained by the method of any one of Embodiments 1-4, for use as an injectable solution for in vivo detection of tumors, preferably PSMA-expressing tumors, by imaging in a subject in need thereof.
6. A solution according to claim 5 wherein the radioactive isotope is selected from the group consisting of ¹¹¹In, ^{133m}In, ^{99m}Tc, ^{94m}Tc, ⁶⁷Ga, ⁶⁶Ga, ⁶⁸Ga, ⁶⁷Ga, ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{177m}Sn, ⁸⁹Zr, ⁴³Sc, ⁴⁴Sc, ⁵⁵Co.
7. The method of any one of Embodiments 1-6, wherein said solution with said radioactive isotope further comprises HCl.
8. The method of any one of Embodiments 1-7, wherein said radioactive isotope is ⁶⁸Ga and the radiochemical purity as measured in HPLC is at least 91%, and optionally, the percentage of free ⁶⁸Ga3+ (in HPLC) is 3% or less, and/or the percentage of non-complexed ⁶⁸Ga3+ species (in ITLC) is 3% or less.
9. The method of any one of Embodiments 1-7, wherein said radioactive isotope is ⁶⁴Cu and the radiochemical purity as measured in HPLC is at least 91%, and optionally, the percentage of free ⁶⁴Cu²⁺ (in HPLC) is 3% or less, and/or the percentage of non-complexed ⁶⁴Cu²⁺ species (in ITLC) is 3% or less.
10. The method of any one of Embodiments 1-7, wherein said radioactive isotope is ⁶⁷Ga and the radiochemical purity as measured in HPLC is at least 91%, and optionally, the percentage of free ⁶⁷Ga3+ (in HPLC) is 3% or less, and/or the percentage of non-complexed ⁶⁷Ga3+ species (in ITLC) is 3% or less.
11. The method of any one of Embodiments 1-10, wherein the incubating step is performed at a temperature between 50° C. to 100° C.
12. The method of any of Embodiments 1-11, wherein the incubating step is performed for a period of time comprised between 2 and 25 minutes.
13. The method of any of Embodiments 1-12, wherein the incubating step is performed at a temperature between 80° C. and 100° C., preferably between 90° C. and 100° C., more preferably at about 95° C.
14. The method of any of Embodiments 1-13, wherein the incubating step is performed for a period of time comprised between 2 and 20 minutes, preferably between 3 and 8 minutes, more preferably about 5 minutes.
15. The method of any of Embodiments 1-12, wherein the incubating step is performed at a temperature between 50° C. and 90° C., preferably between 60° C. and 80° C., more preferably at about 70° C.
16. The method of any of Embodiments 1-13, wherein the incubating step is performed for a period of time comprised between 5 and 25 minutes, preferably between 10 and 20 minutes, more preferably between 12 and 18 minutes, even more preferably about 15 minutes.
17. The method of any one of Embodiments 1-16, wherein said solution of said radioactive isotope is an eluate obtained from the steps of:
   i. producing a radioactive isotope from a parent non-radioactive element by means of a radioactive isotope generator or a cyclotron,
   ii. separating said radioactive isotope from said parent non-radioactive element by elution in HCl as an eluent solvent, and
   iii. recovering the eluate,
thereby obtaining a solution of said radioactive isotope.
18. The method of any one of Embodiments 1-17, wherein said radioactive isotope is ⁶⁸Ga, ⁶⁷Ga or ⁶⁴Cu.
19. The method of any one of Embodiments 1-18, wherein said solution comprising the radioactive isotope ⁶⁸Ga is an eluate obtained from the steps of:
   i. producing ⁶⁸Ga from a parent ⁶⁸Ge by means of a radioactive isotope generator,
   ii. separating the generated ⁶⁸Ga element from ⁶⁸Ge by passing ⁶⁸Ge/⁶⁸Ga through a suitable cartridge, and eluting ⁶⁸Ga in HCl,
thereby obtaining a solution of said radioactive isotope.
20. The method of any one of Embodiments 1-18, wherein said solution comprising the radioactive isotope ⁶⁸Ga is an eluate obtained from the steps of:
   i. producing ⁶⁸Ga from a parent ⁶⁸Zn by means of a cyclotron,
   ii. separating the generated ⁶⁸Ga element from ⁶⁸Zn by passing ⁶⁸Zn/⁶⁸Ga through a suitable cartridge, and eluting ⁶⁸Ga in HCl,
thereby obtaining a solution of said radioactive isotope.
21. The method of any one of Embodiments 1-18, wherein said solution comprising the radioactive isotope ⁶⁷Ga is an eluate obtained from the steps of:
   i. producing ⁶⁷Ga from a parent ⁶⁷Zn or ⁶⁸Zn by means of a cyclotron,
   ii. separating the generated ⁶⁷Ga element from ⁶⁷Zn or ⁶⁸Zn by passing ⁶⁷Zn or ⁶⁸Zn/⁶⁷Ga through a suitable cartridge, and eluting ⁶⁷Ga in HCl,
thereby obtaining a solution of said radioactive isotope
22. The method of any one of Embodiments 1-18, wherein said solution comprising the radioactive isotope ⁶⁴Cu is an eluate obtained from the steps of:
   i. producing ⁶⁴Cu from a parent ⁶⁴Ni by means of a cyclotron,
   ii. separating the generated ⁶⁴Cu element from ⁶⁴Ni by passing ⁶⁴Ni/⁶⁴Cu through a suitable cartridge, and eluting ⁶⁴Cu in HCl,
thereby obtaining a solution of said radioactive isotope
23. A solution comprising PSMA binding ligand of formula (I) labelled with ⁶⁸Ga, ⁶⁷Ga or ⁶⁴Cu, obtainable or obtained by the method of Embodiments 1-22, for use as an injectable solution for in vivo detection of tumors, preferably PSMA-expressing tumors, by imaging in a subject in need thereof
24. A powder for solution for injection, comprising the following components in dried forms:
   i. a PSMA binding ligand of formula (I):

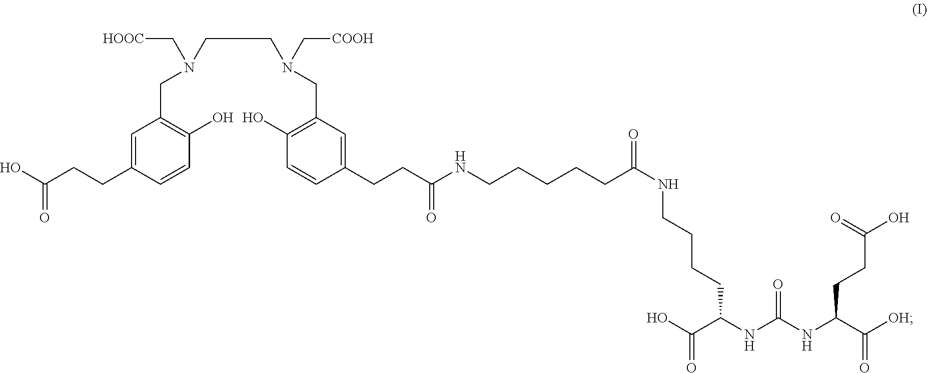 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

ii. sodium chloride;
iii. at least one buffering agent, preferably sodium acetate and;
iv. a stabilizer against radiolytic degradation, preferably gentisic acid.
25. The powder for solution for injection of Embodiments 24, comprising the following components:
   i. a PSMA binding ligand of formula (I) in an amount between 5 μg and 60 μg, preferably between 10 μg and 40 μg, more preferably between 15 μg and 30 μg, even more preferably about 25 μg;

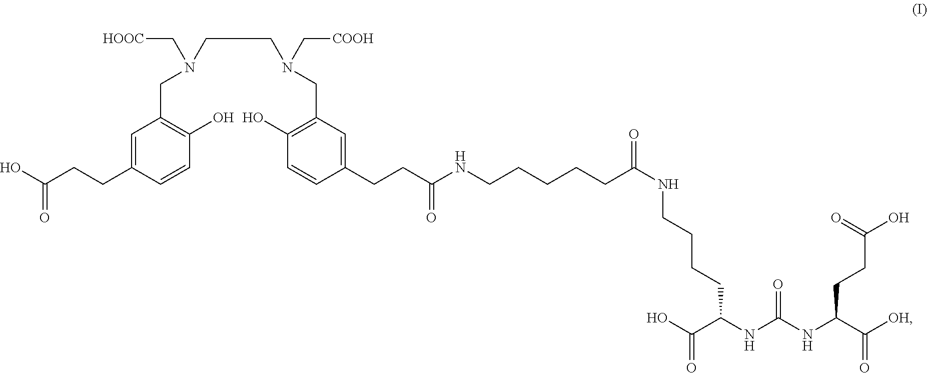 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg and;
iv. gentisic acid in an amount of at least 0.2 mg, preferably between 0.5 mg and 2 mg, more preferably between 0.8 mg and 1.2 mg, even more preferably about 1 mg.
26. The powder for a solution for injection of any of Embodiment 24 or 25, wherein said powder does not contain any bulking agent selected from the group consisting of carbohydrate and polymeric agent, preferably it does not contain any of the following bulking agents: mannitol, maltose, trehalose, polyvinylpyrrolidone and mixtures thereof
27. A kit for carrying out the method of Embodiments 1-22, comprising
   i. a single vial with the following components in dried forms
      i. a PSMA binding ligand of formula (I):

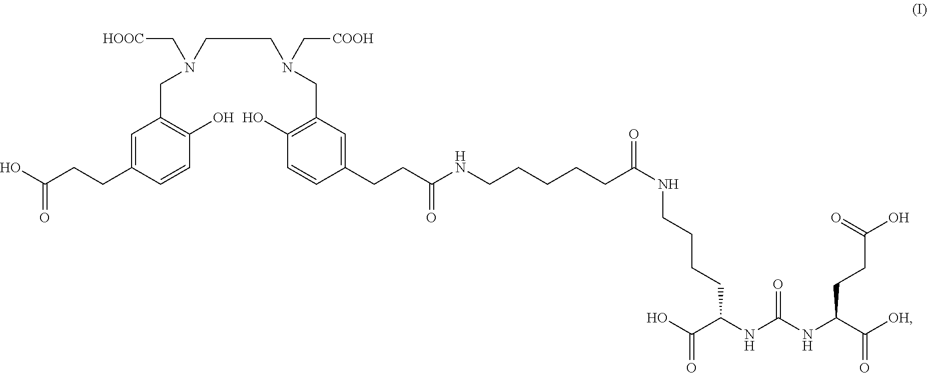 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride;
iii. at least one buffering agent, preferably sodium acetate;
iv. a stabilizer against radiolytic degradation, preferably gentisic acid;
v. optionally, an accessory cartridge for eluting a radioactive isotope generated by a radioactive isotope generator or cyclotron.
28. A kit for carrying out the method of Embodiments 1-22, comprising
   i. a single vial with the following components, preferably in dried forms:
      i. a PSMA binding ligand of formula (I):

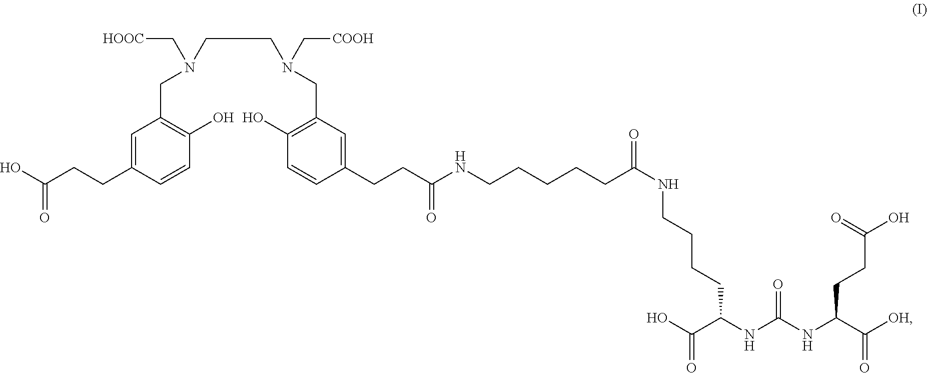 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride;
iii. sodium acetate;
iv. a stabilizer against radiolytic degradation, preferably gentisic acid, and;
v. optionally, an accessory cartridge for eluting a radioactive isotope generated by a radioactive isotope generator or cyclotron.
29. The kit of any one of Embodiment 27 or 28, wherein said single vial comprises, preferably in dried forms, the following components:
   i. a PSMA binding ligand of formula (I) in an amount between 5 μg and 60 μg, preferably between 10 μg and 40 μg, more preferably between 15 μg and 30 μg, even more preferably about 25 μg;

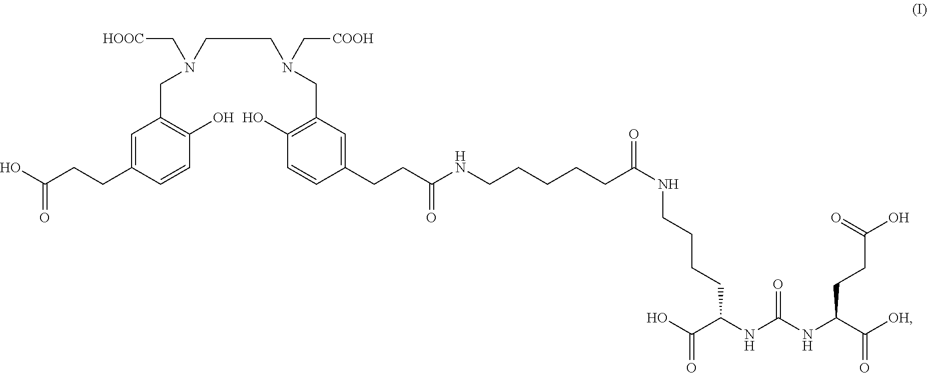 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]

and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg;
iv. gentisic acid in an amount of at least 0.2 mg, preferably between 0.5 mg and 2 mg, more preferably between 0.8 mg and 1.2 mg, even more preferably about 1 mg, and;
v. optionally, an accessory cartridge for eluting a radioactive isotope generated by a radioactive isotope generator or cyclotron.
30. The kit of any one of Embodiments 27-29, wherein said single vial comprises buffering agents suitable for maintaining a pH between 3.0 and 6.0.
31. The kit of any one of Embodiments 27-30, wherein the kit does not contain any bulking agent selected from the group consisting of carbohydrate and polymeric agent, preferably it does not contain any of the following bulking agents: mannitol, maltose, trehalose, polyvinylpyrrolidone and mixtures thereof.

### EXAMPLES

Hereinafter, the present disclosure is described in more details and specifically with reference to the examples, which however are not intended to limit the present invention.

Radiochemical Purity: Non-Complexed ⁶⁸Ga Species by ITLC

iTLC Conditions

| | |
|---|---|
| Method Name: | ⁶⁸GaPSMA-11 |
| TLC Plate type: | ITLC-SG |
| Mobile Phase | AMMONIUM ACETATE (5M): 1 mL |
| | METHANOL: 5 mL |
| | H₂O milliQ: 4 mL |
| Plate Length: | 11.5 cm |
| Sample volume: | 5 μL (1.0 cm from the bottom) |
| Sample run | 6.0 cm (from 1.0 to 7.0 cm) |
| Detector: | Gamma-Beta detector -Raytest |
| Scan Time | 3 minutes |
| Sample Dilution | No dilution |

Radiochemical Purity and Identification of ⁶⁸GaPSMA-11 by HPLC

Chromatographic Conditions

| Flow rate: | 1.000 | mL/min | |
|---|---|---|---|
| Column type: | Aeris 3.6 μm PEPTIDE XB-C18 100 Å, | | |
| | LC Column 150 × 4.6 mm Ea | | |
| Injection volume: | 50 | μL | |
| Detector: | UV-Vis Detector (λ 220 nm) | | |
| Mobile Phase: | A H₂0 FA 0.1%; | | |
| | D ACN FA 0.1%. | | |
| | Time | | |
| Program: | (min) | A (%) | D (%) |
| | 0.000 | 90% | 10% |
| | 0.500 | 90% | 10% |
| | 16.000 | 75% | 25% |
| | 16.000 | 40% | 60% |
| | 18.000 | 40% | 60% |
| | 18.000 | 90% | 10% |
| | 21.000 | 90% | 10% |

| Column temp: | 25° C. | |
|---|---|---|
| Detector time: | 18.000 | min |
| Run time: | 21.000 | min |

### Example 1: Development of a Method for Radiolabeling PSMA-11 with ⁶⁸Ga Using a Single Vial Kit

1. Description and Composition of the Single Vial Kit

The Applicant developed a sterile single vial kit which consists of:
A single vial: PSMA-11, 25 μg; sodium chloride, 40 mg; sodium acetate buffer, 47 mg; gentisic acid antioxidant, 1 mg. All in dried form, powder for solution for injection, to be reconstituted with a solution of gallium-68 chloride (⁶⁸GaCl₃) in HCl eluted from a ⁶⁸Ge/⁶⁸Ga generator.

The kit is used in combination with a solution of ⁶⁸Ga in dilute HCl eluted from a ⁶⁸Ge/⁶⁸Ga generator to prepare ⁶⁸Ga-PSMA-11 as radiolabelled imaging product for intravenous injection.

The volume of the ⁶⁸Ga-PSMA-11 solution for injection, corresponding to the radioactive dose to be administered, is calculated according to the estimated time of injection, on the basis of the current activity provided by the generator and of physical decay of the radionuclide (half-life=68 min).

The single vial is a powder for solution for injection containing 25 μg PSMA-11 as active ingredient, packed in 10 mL Ultra inert Type I Plus glass vials.

The composition of the single vial is provided in Table 1.

TABLE 1
Composition of the single vial powder for solution for injection

| | Composition | | |
|---|---|---|---|
| Component | (per vial) | Function | Quality Standard |
| PSMA-11 | 25 μg | Active substance | In-house |
| Sodium chloride | 40 mg | | Ph.Eur/USP* |
| Sodium acetate | 47 mg | Buffer agent | Ph.Eur/USP* |
| Gentisic acid | 1 mg | Antioxydant | Ph.Eur/USP* |

*current version

As described above, the single vial (PSMA-11, 25 μg, powder for solution for injection) is part of a radiopharmaceutical kit. The kit has to be used in combination with a solution of ⁶⁸Ga in HCl provided by a ⁶⁸Ge/⁶⁸Ga generator to obtain ⁶⁸Ga-PSMA-11 solution for injection, being the Radiolabelled Imaging Product, which can be directly injected to the patient.

2. Components of the Drug Product

The drug product contains PSMA-11 as active ingredient and Sodium chloride, Sodium acetate and Gentisic acid as excipient.

2.1 Drug Substance

The active substance is the PSMA-11 peptide, a Lys-ureido-Glu sequence covalently bound to a chelator HBED-CC (N,N′-bis[2-hydroxy-5-(carboxyethyl)benzyl]-ethylenediamine-N,N′-diacetic acid) at the lysine end through a spacer molecule. The formula (I) of the PSMA-11 is as follows:

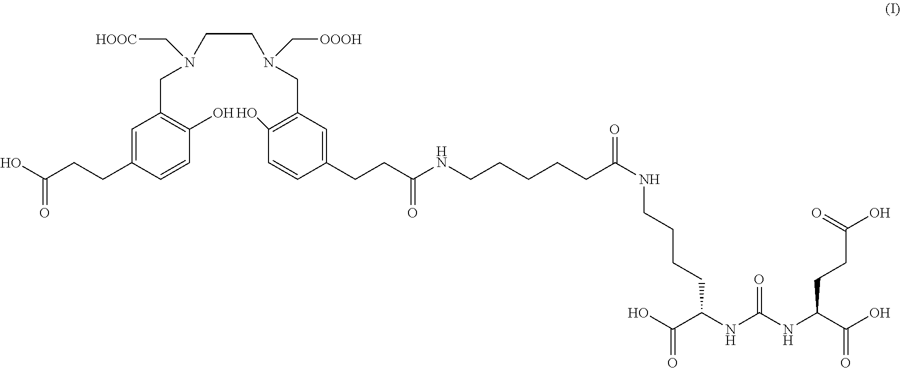

The HBED-CC is the moiety of the API that can chelate the ⁶⁸Ga and allow PSMA-11 to work as a tracer for Prostate Cancer imaging. The formula (II) of the ⁶⁸Ga-PSMA-11 is as follows:

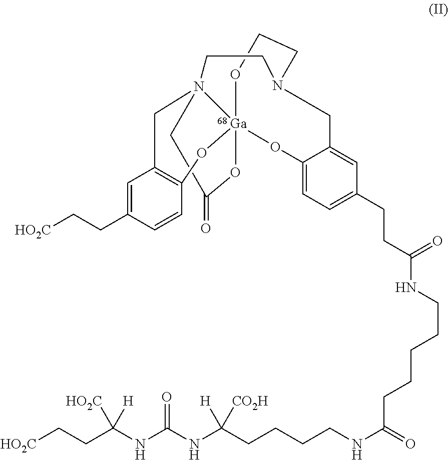

2.2 Excipients

The excipients chosen for the composition of the single vial are added to maintain stability of the active substance in the final formulation, to assure safety and efficacy of the drug product and also to obtain the required radiochemical purity of the ⁶⁸Ga-PSMA-11 solution during the reconstitution procedure. The excipients selected lead to a drug product with the required pharmaco-technical characteristics.

A brief description of each excipient is provided as follows:

Sodium Acetate

Sodium acetate is used as a buffer agent. Buffers are chemically defined as solutions containing either a weak acid and its conjugated salt or a weak base and its conjugated salt. Buffers are commonly used to maintain pH within a certain range as they can neutralize small quantities of addition acid or base.

The reason for including a Buffer Agent in the PSMA-11 Kit formulation was to have a one vial kit able to maintain the pH within a range that allows the complete complexation of ⁶⁸Ga in the HBED moiety.

Sodium Chloride

Sodium chloride is used for the solubility, integrity of the cake and product stability. The reason for including sodium chloride in the PSMA-11 Kit formulation was to allow operating extreme conditions during the freeze drying without affecting the properties of the formulation.

Gentisic Acid

Gentisic acid (2,5-Dihydroxybenzoic acid) has been found here as highly effective antioxidant or radical scavengers or stabilizer against radiolytic degradation. This substance is used to extend the shelf-life of medicines by retarding oxidation of active substances. Specifically, it is included in radiopharmaceuticals because they allow protecting an API from radiolysis.

3. Drug Product

3.1 Formulation Development

The formulation development has been performed with the aim of identifying the reaction mixture composition able to allow:
the production of an acceptable cake after the freeze-drying, and
a simple labeling of the HBED-CC-molecule based on direct reconstitution with the eluate from commercially available 68Ge/68Ga generators without any processing of the eluate or any additional purification step.

The goal of this project was to develop the PSMA-11 small molecule to be used as radiotracer for the detection of prostate tumors.

The single vial is a lyophilisate powder containing the peptide as active ingredient which is radiolabelled with ⁶⁸Ga during the radiolabeling procedure.

Initial efforts to develop a suitable formulation for PSMA-11 have involved tests in liquid form.

The drug product manufacturer focused the development work on the selection of the appropriate excipients in relation with the PSMA-11 characteristics in order to obtain a finished product meeting the specifications commonly required for radiopharmaceutical preparations
⁶⁸Ga-PSMA-11 (HPLC): ≥91%
Free ⁶⁸Ga³⁺ (HPLC): ≤3%
Non-complexed ⁶⁸Ga³⁺ species (ITLC): ≤3%

The development work including the relevant performed studies is described starting from the selection of the active ingredient amount and appropriate excipients.

3.1.1 Selection of the PSMA-11 Amount

Tests, using different amounts of API, were performed in order to select the quantity of PSMA-11 for the drug product.

The rationale for the preliminary tests ware based on the Draft of GALLIUM (⁶⁸Ga) PSMA-11 INJECTION monograph (3044). Even though this monograph is not yet included in the current European Pharmacopeia it is already available on Pharmeuropa, which is a free online EDQM (European Directorate for the Quality of Medicines and Healthcare) publication providing public inquiries on draft European texts. In the monograph 3044, the maximum recommended dose is 30 μg of PSMA-11; hence, preliminary tests were carried out using this amount of API. The aim of these tests was to confirm that 30 μg of PSMA-11 were sufficient to consistently produce [68Ga]Gallium PSMA-11 with high radiochemical purity. Once demonstrated that 30 μg were a reliable amount for radiolabeling of PSMA-11 using three different generator (Gallipharm, GalliAd, ITG), lower amounts were tested, in order to comprehend whether it was possible to reduce the API content without any effect on the radiochemical purity of the product. Specifically, lowest amounts of PSMA-11 (20 μg and 10 μg) were tested only in the more diluted condition (Galliapharm—5 mL) as higher concentration of reagents promote and facilitate the reaction.

The final amount of API selected for the development of PSMA-11 Kit was 25 μg. Even though lower amount (20 μg) demonstrated to be enough for a consistently radiolabeling of PSMA-11 with ⁶⁸Ga highly above the radiochemical purity expectations as well, 25 μg was conserved as a safe quantity for future use of the product with ⁶⁸Ga from Cyclotron.

TABLE 2
PSMA-11 amount-effects on the amount of PSMA-11 on RCP%

| PSMA-11 | | | | |
|---|---|---|---|---|
| Amount | | | | Radiochemical |
| (μg) | | Radiochemical Purity by HPLC | | Purity by iTLC |
| (47 mg of | | [⁶⁸Ga] | [⁶⁸Ga] Gallium | [⁶⁸Ga] Not |
| Sodium | Volume | Gallium | PSMA-11 | Complexed |
| Acetate) | (mL) | free | (sum of stereosiomers) | Species |
| 30 | 5 | 0.13% | 99.69% | 0.00% |
| 30 | 4 | 0.00% | 99.04% | 0.00% |
| 30 | 1 | 0.03% | 99.72% | 0.00% |
| 25 | 5 | 0.41% | 99.11% | 0.00% |
| 25 | 4 | 0.00% | 98.93% | 0.00% |
| 25 | 1 | 0.15% | 99.44% | 0.00% |
| 20 | 5 | 0.00% | 100% | 0.00% |

Our development study was also focused on the selection of critical excipients.

3.1.2 Excipients

Selection of the Buffer Agent

Buffers are commonly used to maintain pH within a certain range as they can neutralize small quantities of addition acid or base. pH range is defined as the pH space within the buffer agent works and can carry out its buffering ability. The pH range is strictly related to the chemical entity of the weak acid or the weak base of the buffer.

Since the chelating agent HBED represents the compound moiety involved for the radiolabeling reaction, the target pH range was set at 4.0-6.0. The latter was selected accordingly to the literature of HBED and previous works of ⁶⁸Ga PSMA-11.

The list of Buffer Agents commonly used in pharmaceutical injectable solutions able to carry out their buffering capabilities in the selected range are Citrate Buffer, Lactate Buffer, Acetate Buffer, Phosphate Buffer and Glycine Buffer.

However, the final product has to be freeze-dried. There is, indeed, a broad literature describing which excipients are more compliant with the lyophilisation process and easier to be freeze dried. Based on the data and the experience collected from previous work and products, the Buffer Agents suitable for freeze dried products are Glycine, Sodium Citrate, Sodium Lactate and Sodium Phosphate.

Tests were performed radiolabeling 25 μg of PSMA-11 with the suitable amount of each salt and eluting an E&Z Generator (5 mL HCl 0.1 M) in order to simulate the lowest pH condition for radiolabeling.

TABLE 3
Buffer Agent Tests

| Buffer | | | | | |
|---|---|---|---|---|---|
| Agent | | | Radiochemical Purity by iTLC | | |
| amount | | | | [⁶⁸Ga] Gallium | |
| (PSMA- | | | [⁶⁸Ga] | PSMA-11 | [⁶⁸Ga] Not |
| 11 25 | Generator/ | | Gallium | (sum of | Complexed |
| μg) | Activity | pH | free | stereosiomers) | Species |
| Sodium | IRE | 5.42 | 100% | 0.0% | 100% |
| Citrate | 15.32 mCi | | | | |
| 104 mg | | | | | |
| Sodium | E&Z | 3.36 | 3.62% | 11.51% | 56.22% |
| Lactate | 16.83 mCi | | | | |
| 93 mg | | | | | |
| Na₂HPO₄ | IRE | 7.15 | 3.56% | 80.55% | 5.85% |
| 71 mg | 17.12 mCi | | | | |
| Sodium | IRE | 5.25 | 0.00% | 99.60% | 0.00% |
| Acetate | 17.02 mCi | | | | |
| 47 mg | | | | | |

Data collected showed that the only Sodium Salt compliant with the radiolabeling procedure was Sodium Acetate.

Different amounts were investigated in order to evaluate the robustness of the radiochemical purity at different amounts of Sodium Acetate. Tests were performed radiolabeling 25 μg of PSMA-11 with different amounts of Sodium Acetate.

TABLE 4
Buffer Agent quantity tests

| Sodium | | | | | |
|---|---|---|---|---|---|
| Acetate | | | | | |
| amount | | | Radiochemical Purity by iTLC | | |
| (PSMA- | | | [⁶⁸Ga] | [⁶⁸Ga] Gallium | [⁶⁸Ga] Not |
| 11 25 | Generator/ | | Gallium | PSMA-11 | Complexed |
| μg) | Activity | pH | free | (sum of stereosiomers) | Species |
| Sodium | E&Z | 1.91 | 3.79% | 75.43% | 6.58% |
| Acetate | 11.50 mCi | | | | |
| 40 mg | | | | | |
| Sodium | E&Z | 3.92 | 0.00% | 99.90% | 0.00% |
| Acetate | 14.52 mCi | | | | |
| 47 mg | | | | | |
| Sodium | IRE | 5.27 | 0.07% | 99.66% | 0.00% |
| Acetate | 18.13 mCi | | | | |
| 47 mg | | | | | |
| Sodium | IRE | 5.28 | 0.10% | 99.03% | 0.00% |
| Acetate | 18.49 mCi | | | | |
| 50 mg | | | | | |

Based on the data collected, 47 mg resulted to be the minimum quantity of sodium Acetate able to produce ⁶⁸Ga PSMA-11 with a radiochemical purity highly above the expectations.

Selection of the Antioxidant

Radical Scavenger to be used for the improvement of the radiochemical stability of ⁶⁸Ga-PSMA-11, tests were performed according to a varied approach in order to consider all the parameters that may affect the radiochemical purity of the product (activity, pH, volume, amount of Gentisic Acid).

Based on the data collected, 1000 μg of Gentisic Acid resulted to be the suitable amount of antioxidant agent capable of protecting ⁶⁸GaPSMA-11 from radiolysis in all the radiochemical stability conditions tested. Even though lower amounts (400 μg) demonstrated to allow ⁶⁸Ga-PSMA-11 being stable up to 4 hours with high radiochemical purity results, 1000 μg was conserved as a safe quantity for future use of the product with ⁶⁸Ga from Cyclotron.

TABLE 5
Radiochemical Stability Data with Gentisic Acid

| | | | | Radiochemical Purity at t0 h | | | | Radiochemical Purity at t4 h | | | |
|---|---|---|---|---|---|---|---|---|---|---|---|
| | | | | HPLC | | | | HPLC | | | |
| | | | | | | [⁶⁸Ga] | iTLC | | | [⁶⁸Ga] | iTLC |
| | | | Gentisic | | [⁶⁸Ga] | Gallium | [⁶⁸Ga] | | [⁶⁸Ga] | Gallium | [⁶⁸Ga] |
| | Stability | | Acid | [⁶⁸Ga] | Gallium | PSMA-11 | Not | [⁶⁸Ga] | Gallium | PSMA-11 | Not |
| Generator/ | Volume | | Amount | Gallium | main | (sum of | Complexed | Gallium | main | (sum of | Complexed |
| Activity | mL | pH | (μg) | free | degradant | stereosiomers) | Species | free | degradant | stereosiomers) | Species |
| IRE | 1 | 5.24 | 0 | 0.15% | 6.61% | 92.71% | 0.00% | 0.14% | 7.32% | 90.89% | 0.00% |
| 17.19 | | | | | | | | | | | |
| mCi | | | | | | | | | | | |
| IRE | 1 | 5.25 | 200 | 0.00% | 1.21% | 98.46% | 0.00% | 0.00% | 1.74% | 97.80% | 0.00% |
| 18.07 | | | | | | | | | | | |
| mCi | | | | | | | | | | | |
| IRE | 1 | 5.22 | 200 | 0.00% | 1.83% | 97.76% | 0.23% | 0.00% | 1.80% | 97.87% | 0.00% |
| 17.85 | | | | | | | | | | | |
| mCi | | | | | | | | | | | |
| ITG | 4 | 4.83 | 200 | 0.00% | 0.19% | 99.56% | 0.00% | 0.00% | 0.00% | 99.72% | 0.00% |
| 35.92 | | | | | | | | | | | |
| mCi | | | | | | | | | | | |
| IRE | 1 | 5.11 | 400 | 0.12% | 1.00% | 98.88% | 0.00% | 0.00% | 1.54% | 98.46% | 0.00% |
| 17.79 | | | | | | | | | | | |
| mCi | | | | | | | | | | | |
| IRE | 1 | 5.13 | 400 | 0.00% | 0.71% | 99.29% | 0.00% | 0.00% | 0.68% | 99.20% | 0.00% |
| 17.44 | | | | | | | | | | | |
| mCi | | | | | | | | | | | |
| ITG | 4 | 4.85 | 400 | 0.08% | 0.19% | 99.16% | 0.00% | 0.00% | 0.00% | 98.24% | 0.00% |
| 16.9 | | | | | | | | | | | |
| mCi | | | | | | | | | | | |
| ITG | 4 | 4.81 | 400 | 0.00% | 0.16% | 99.35% | 0.00% | 0.00% | 0.00% | 99.57% | 0.00% |
| 31.97 | | | | | | | | | | | |
| mCi | | | | | | | | | | | |
| IRE | 1 | 5.08 | 1000 | 0.00% | 0.33% | 99.67% | 0.00% | 0.00% | 0.41% | 99.59% | 0.00% |
| 17.1 | | | | | | | | | | | |
| mCi | | | | | | | | | | | |
| ITG | 4 | 4.87 | 1000 | 0.01% | 0.02% | 99.80% | 0.00% | 0.00% | 0.06% | 99.69% | 0.00% |
| 30.9 | | | | | | | | | | | |
| mCi | | | | | | | | | | | |
| E&Z | 5 | 3.75 | 1000 | 0.00% | 0.00% | 99.87% | 0.00% | 0.00% | 0.00% | 100.00% | 0.00% |
| 15.65 | | | | | | | | | | | |
| mCi | | | | | | | | | | | |

Selection of Sodium Chloride

One of the aims of a freeze-drying process is to obtain a pharmaceutically elegant end product while retaining the activity of the API. This goal is usually achieved by adding excipients such as Bulking agents which are crystalline materials capable of offering a robust matrix so that the primary drying can be conducted at high temperatures.

Once defined the suitable amounts of API and Sodium Acetate required for the development of PSMA-11 Kit, tests were performed in order to evaluate whether the addition of a Bulking Agent was necessary to improve the elegance of the final cake.

The extensive approach was performed freeze drying, through the most conservative process conditions, different formulations containing the same amount of Sodium Acetate (47 mg) with different Bulking Agents (Mannitol, Trehalose and PVP) at different concentrations (30 and 90 mg/mL). The API was not included in these extensive tests as its amount was so low, compared to the Sodium Acetate, that it was ignored. The products were manufactured by the extensive approach with and without mannitol.

Other tests have been carried out using other bulking agents as Trehalose, Maltose, Sucrose, PVP and their combinations. However results collected from the investigation demonstrated that bulking agents tested were not capable to prevent the cake from collapse when the process of freeze-drying was more extreme.

Another approach was applied to test for the first time the combination of sodium acetate with crystalline salts as KCl and NaCl. The formulations containing Sodium Chloride resulted in very elegant cakes.

Considering the appearance of the cakes manufacture with 40 mg of Sodium Chloride, these products were tested for radiolabeling. Data collected from the quality controls performed on the ⁶⁸Ga radiolabelled product are shown in Table 6.

TABLE 6
Radiolabeling tests of kits manufactured with: 25 μg PSMA-11, 47 mg
Sodium Acetate, 40 mg Sodium Chloride.

| | | | | | Radiochemical |
|---|---|---|---|---|---|
| | | | | | Purity by |
| | | | Radiochemical Purity by HPLC | | iTLC |
| | | | [⁶⁸Ga] | [⁶⁸Ga] Gallium | [⁶⁸Ga] Not |
| Generator/ | Volume | | Gallium | PSMA-11 | Complexed |
| Activity | mL | pH | free | (sum of stereosiomers) | Species |
| IRE | 1 | 5.12 | 0.00% | 99.40% | 0.00% |
| 13.19 mCi | | | | | |
| IRE | 1 | 5.19 | 0.00% | 97.81% | 0.00% |
| 18.19 mCi | | | | | |
| ITG | 4 | 4.83 | 0.00% | 99.52% | 0.00% |
| 35.73 mCi | | | | | |
| ITG | 4 | 4.83 | 0.00% | 99.53% | 0.00% |
| 36.17 mCi | | | | | |

Batches produced during the development stages demonstrated that the manufacturing of a freeze dried product intended as a kit for preparation of [68Ga]Gallium PSMA-11 was not achievable when Bulking Agents were included in the formulations. Limits for adding a Bulking Agent were multiple: solubility, integrity of the cake and product stability. Nevertheless, when crystalline Sodium Chloride was added to the formulation, all the limiting issues were overcome. Based on the experience achieved during the preliminary batches, Bulking Agents were excluded as formulation components and Sodium Chloride was included.

According to the products manufactured with crystalline salts, it was observed that the defect of the end products manufactured when 47 mg of Sodium Acetate were mixed with Sodium Chloride was the lifting of the cake from the bottom of the vial. Since this defect was in percentage more frequent in the kits containing only 20 mg of NaCl than those with 40 mg it was necessary to comprehend which was the quantity of the Sodium Chloride capable of either preventing or reducing the cake lift. Another batch was then manufactured in order to investigate four level of Sodium Chloride:
1. 20 mg
2. 40 mg
3. 60 mg
4. 80 mg

In this case, the freeze drying cycle was set in order to manufacture the products through extreme conditions. Data collected resulted to be very promising. Despite the severe parameters of the process, most of the cakes kept the elegance observed in the previous batch and it was clear that above 40 mg of Sodium Chloride the positive effect of reducing the product lift became not relevant.

4. Radiolabeling Procedure

Based on the single kit design, a 2-step labeling procedure has been developed as follows:
1. direct reconstitution of the powder vial with the solution of ⁶⁸Ga in HCl provided by the ⁶⁸Ge/⁶⁸Ga E&Z generator.
2. incubation of the reaction at room temperature, between 18° C. and 25° C. for 5 minutes.

At this point the ⁶⁸Ga-PSMA-11 solution is ready for administration.

5. Final Formulation and Detailed Composition

Based on all development performed on the formulation as above presented, the final chosen formulation of the single vial is the following:

TABLE 7
Final formulation

| | Quality Standard (and | | Quantity |
|---|---|---|---|
| Component | Grade, if applicable) | Purpose | per vial |
| PSMA-11 | In-house | Active substance | 25 μg |
| Sodium chloride | Ph.Eur/USP* | Crystalline salt | 40 mg |
| Sodium acetate | Ph.Eur/USP* | Buffer agent | 47 mg |
| Gentisic acid | Ph.Eur/USP* | Antioxydant | 1 mg |
| | | agent | |

*current version

Stability tests were performed on the freeze dried product containing the final formulation. Data collected showed very promising results in terms of the shelf-life of the Drug Product. Kits manufactured with the final formulation, indeed, were stored at different stability conditions (2-8° C.; 25° C. and 40° C.) and were tested at different time points for API-assay, Radiochemical Purity and Radiochemical Stability of the radiolabelled product. Radiochemical Purity and Stability were assessed only eluting IRE Generator which represented the most stressful condition in terms of Radioactive Concentration (Volumic Activity).

TABLE 8
API Assay Stability Data at Manufacturing date
API quantitation based on the external calibration curve

| Time Point | 1. injection | 2. injection | 3. injection | Average |
|---|---|---|---|---|
| Release | 28.31 μg | 28.76 μg | 27.40 μg | 28.15 μg |

TABLE 9
Radiochemical Purity and Radiochemical Stability Data at Manufacturing date

| | | | | Radiochemical Purity at t0 h | | | | Radiochemical Purity at t4 h | | | |
|---|---|---|---|---|---|---|---|---|---|---|---|
| | | | | HPLC | | | | HPLC | | | |
| | | | | | | [⁶⁸Ga] | iTLC | | | [⁶⁸Ga] | iTLC |
| | | | | | [⁶⁸Ga] | Gallium | [⁶⁸Ga] | | [⁶⁸Ga] | Gallium | [⁶⁸Ga] |
| | | Stability | | [⁶⁸Ga] | Gallium | PSMA-11 | Not | [⁶⁸Ga] | Gallium | PSMA-11 | Not |
| Time | Generator/ | Volume | | Gallium | main | (sum of | Complexed | Gallium | main | (sum of | Complexed |
| Point | Activity | mL | pH | free | degradant | stereosiomers) | Species | free | degradant | stereosiomers) | Species |
| Release | IRE | 1 | 5.07 | 0.00% | 0.23% | 99.66% | 0.00% | 0.03% | 0.42% | 99.05% | 0.00% |
| | 32.95 | | | | | | | | | | |
| | mCi | | | | | | | | | | |
| | IRE | 1 | 4.95 | 0.00% | 0.00% | 99.58% | 0.00% | 0.00% | 0.54% | 98.75% | 0.00% |
| | 32.27 | | | | | | | | | | |
| | mCi | | | | | | | | | | |
| | E&Z | 5 | 3.75 | 0.00% | 0.00% | 99.87% | 0.00% | 0.00% | 0.00% | 100.00% | 0.00% |
| | 15.65 | | | | | | | | | | |
| | mCi | | | | | | | | | | |
| | E&Z | 5 | 3.75 | 0.00% | 0.19% | 99.68% | 0.51% | 0.00% | 0.00% | 100.00% | 0.00% |
| | 16.80 | | | | | | | | | | |
| | mCi | | | | | | | | | | |
| | ITG | 4 | 4.67 | 0.01% | 0.02% | 98.80% | 0.00% | 0.00% | 0.06% | 99.69% | 0.00% |
| | 30.90 | | | | | | | | | | |
| | mCi | | | | | | | | | | |
| | ITG | 4 | 4.67 | 0.00% | 0.05% | 99.81% | 0.00% | 0.00% | 0.00% | 99.82% | 0.00% |
| | 29.74 | | | | | | | | | | |
| | mCi | | | | | | | | | | |

TABLE 10
API Assay Stability Data at 2-8° C.
API quantitation based on the external calibration curve

| Time Point | 1. injection | 2. injection | 3. injection | Average |
|---|---|---|---|---|
| 3 months | 28.87 μg | 29.21 μg | 28.44 μg | 28.84 μg |
| 6 months | 29.79 μg | 29.61 μg | 29.43 μg | 29.61 μg |

TABLE 11
Radiochemical Purity and Radiochemical Stability Data at 2-8° C.

| | | | | Radiochemical Purity at t0 h | | | | Radiochemical Purity at t4 h | | | |
|---|---|---|---|---|---|---|---|---|---|---|---|
| | | | | HPLC | | | | HPLC | | | |
| | | | | | | [⁶⁸Ga] | iTLC | | | [⁶⁸Ga] | iTLC |
| | | | | | [⁶⁸Ga] | Gallium | [⁶⁸Ga] | | [⁶⁸Ga] | Gallium | [⁶⁸Ga] |
| | | Stability | | [⁶⁸Ga] | Gallium | PSMA-11 | Not | [⁶⁸Ga] | Gallium | PSMA-11 | Not |
| Time | Generator/ | Volume | | Gallium | main | (sum of | Complexed | Gallium | main | (sum of | Complexed |
| Point | Activity | mL | pH | free | degradant | stereosiomers) | Species | free | degradant | stereosiomers) | Species |
| 3 | IRE | 1 | 5.29 | 0.04% | 0.55% | 99.28% | 0.00% | 0.00% | 0.49% | 99.37% | 0.00% |
| months | 25.76 | | | | | | | | | | |
| | mCi | | | | | | | | | | |
| 6 | IRE | 1 | 5.13 | 0.00% | 0.47% | 99.40% | 0.00% | 0.00% | 0.44% | 99.44% | 0.00% |
| months | 19.90 | | | | | | | | | | |
| | mCi | | | | | | | | | | |

TABLE 12
API Assay Stability Data at 25° C.
API quantitation based on the external calibration curve

| Time Point | 1. injection | 2. injection | 3. injection | Average |
|---|---|---|---|---|
| 3 months | 28.19 μg | 29.57 μg | 28.33 μg | 28.70 μg |
| 6 months | 29.35 μg | 29.31 μg | 29.06 μg | 29.24 μg |

TABLE 13
Radiochemical Purity and Radiochemical Stability Data at 25° C.

| | | | | Radiochemical Purity at t0 h | | | | Radiochemical Purity at t4 h | | | |
|---|---|---|---|---|---|---|---|---|---|---|---|
| | | | | HPLC | | | | HPLC | | | |
| | | | | | | [⁶⁸Ga] | iTLC | | | [⁶⁸Ga] | iTLC |
| | | | | | [⁶⁸Ga] | Gallium | [⁶⁸Ga] | | [⁶⁸Ga] | Gallium | [⁶⁸Ga] |
| | | Stability | | [⁶⁸Ga] | Gallium | PSMA-11 | Not | [⁶⁸Ga] | Gallium | PSMA-11 | Not |
| Time | Generator/ | Volume | | Gallium | main | (sum of | Complexed | Gallium | main | (sum of | Complexed |
| Point | Activity | mL | pH | free | degradant | stereosiomers) | Species | free | degradant | stereosiomers) | Species |
| 3 | IRE | 1 | 5.16 | 0.05% | 0.19% | 99.22% | 0.00% | 0.00% | 0.38% | 99.03% | 0.00% |
| months | 24.56 | | | | | | | | | | |
| | mCi | | | | | | | | | | |
| 6 | IRE | 1 | 5.09 | 0.00% | 0.42% | 99.32% | 0.00% | 0.00% | 0.33% | 99.21% | 0.00% |
| months | 18.88 | | | | | | | | | | |
| | mCi | | | | | | | | | | |

TABLE 14
API Assay Stability Data at 40° C.
API quantitation based on the external calibration curve

| Time Point | 1. injection | 2. injection | 3. injection | Average |
|---|---|---|---|---|
| 3 months | 28.17 μg | 28.05 μg | 28.06 μg | 28.10 μg |
| 6 months | 28.81 μg | 28.87 μg | 28.98 μg | 28.89 μg |

TABLE 15
Radiochemical Purity and Radiochemical Stability Data at 40° C.

| | | | | Radiochemical Purity at t0 h | | | | Radiochemical Purity at t4 h | | | |
|---|---|---|---|---|---|---|---|---|---|---|---|
| | | | | HPLC | | | | HPLC | | | |
| | | | | | | [⁶⁸Ga] | iTLC | | | [⁶⁸Ga] | iTLC |
| | | | | | [⁶⁸Ga] | Gallium | [⁶⁸Ga] | | [⁶⁸Ga] | Gallium | [⁶⁸Ga] |
| | | Stability | | [⁶⁸Ga] | Gallium | PSMA-11 | Not | [⁶⁸Ga] | Gallium | PSMA-11 | Not |
| Time | Generator/ | Volume | | Gallium | main | (sum of | Complexed | Gallium | main | (sum of | Complexed |
| Point | Activity | mL | pH | free | degradant | stereosiomers) | Species | free | degradant | stereosiomers) | Species |
| 3 | IRE | 1 | 5.23 | 0.07% | 0.26% | 98.92% | 0.00% | 0.00% | 0.37% | 98.20% | 0.00% |
| months | 24.86 | | | | | | | | | | |
| | mCi | | | | | | | | | | |
| 6 | IRE | 1 | 5.15 | 0.04% | 0.42% | 99.21% | 0.00% | 0.00% | 0.46% | 99.23% | 0.00% |
| months | 19.34 | | | | | | | | | | |
| | mCi | | | | | | | | | | |

1. A method for labeling a PSMA binding ligand with a radioactive isotope, preferably ⁶⁸Ga, ⁶⁷Ga or ⁶⁴Cu, said method comprising the steps of:
i. providing a single vial comprising, in dried form, said PSMA binding ligand of the following formula (I): 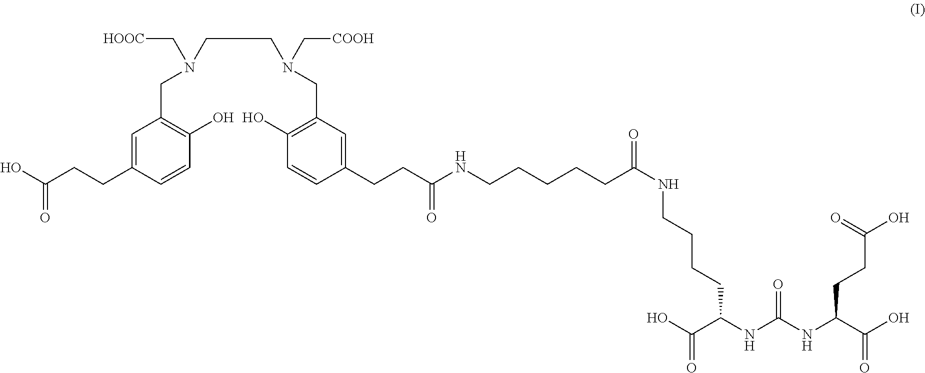 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
at least one buffering agent, sodium chloride and a stabilizer against radiolytic degradation,
ii. adding a solution of said radioactive isotope into said single vial, thereby obtaining a solution of said PSMA binding ligand of formula (I) with said radioactive isotope,
iii. mixing the solution obtained in ii., and incubating it for a sufficient period of time for obtaining said PSMA binding ligand labelled with said radioactive isotope, and,
iv. optionally, adjusting the pH of the solution.

2. The method of claim 1, wherein said at least one buffering agent is sodium acetate.

3. The method of claim 1, wherein said single vial does not contain any bulking agent selected from the group consisting of carbohydrate and polymeric agent, preferably it does not contain any of the following bulking agents: mannitol, maltose, trehalose, polyvinylpyrrolidone, and mixtures thereof and the buffering agent is a buffer suitable for obtaining a pH from 3.0 to 6.0, at the incubating step (iii).

4. The method of claim 1, wherein said PSMA binding ligand of formula (I) is comprised in said single vial in an amount between 5 μg and 60 μg, preferably between 10 μg and 40 μg, more preferably between 15 μg and 30 μg, even more preferably about 25 μg.

5. A solution comprising a PSMA binding ligand of formula (I) labelled with a radioactive isotope, obtainable or obtained by the method of claim 1, for use as an injectable solution for in vivo detection of tumors, preferably PSMA-expressing tumors, by imaging in a subject in need thereof.

6. A solution according to claim 5 wherein the radioactive isotope is selected from the group consisting of ¹¹¹In, ^{133m}In, ^{99m}Tc, ^{94m}Tc, ⁶⁷Ga, ⁶⁶Ga, ⁶⁸Ga, ⁶⁷Ga ⁵²Fe, ⁷²As, ⁹⁷Ru, ²⁰³Pb, ⁶²Cu, ⁶⁴Cu, ⁸⁶Y, ⁵¹Cr, ^{52m}Mn, ¹⁵⁷Gd, ¹⁶⁹Yb, ¹⁷²Tm, ^{177m}Sn, ⁸⁹Zr, ⁴³Sc, ⁴⁴Sc, ⁵⁵Co.

7. A solution comprising PSMA binding ligand of formula (I) labelled with ⁶⁸Ga, ⁶⁷Ga or ⁶⁴Cu obtainable or obtained by the method of claim 1, for use as an injectable solution for in vivo detection of tumors, preferably PSMA-expressing tumors, by imaging in a subject in need thereof.

8. A powder for a solution for injection, comprising the following components in dried forms:
i. a PSMA binding ligand of formula (I): 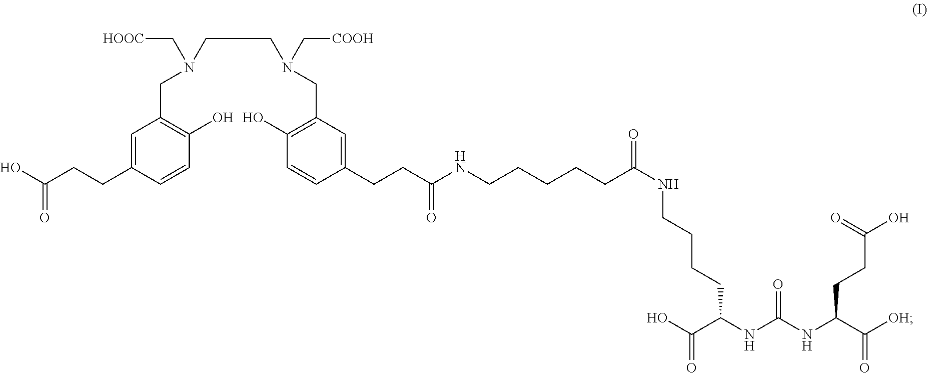 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
ii. sodium chloride;
iii. at least one buffering agent, preferably sodium acetate and;
iv. a stabilizer against radiolytic degradation, preferably gentisic acid.

9. The powder for a solution for injection of claim 8, comprising the following components:
i. a PSMA binding ligand of formula (I) in an amount between 5 μg and 60 μg, preferably between 10 μg and 40 μg, more preferably between 15 μg and 30 μg, even more preferably about 25 μg; 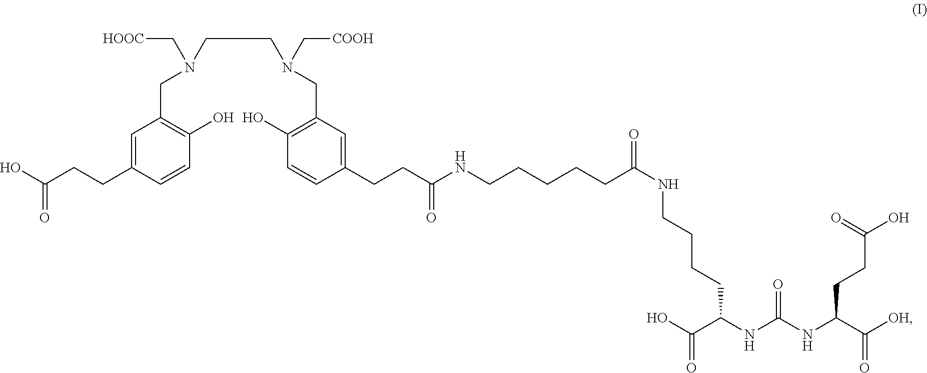 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg and;
iv. gentisic acid in an amount of at least 0.2 mg, preferably between 0.5 mg and 2 mg, more preferably between 0.8 mg and 1.2 mg, even more preferably about 1 mg.

10. The powder for a solution for injection of claim 8, wherein said powder does not contain any bulking agent selected from the group consisting of carbohydrate and polymeric agent, preferably it does not contain any of the following bulking agents: mannitol, maltose, trehalose, polyvinylpyrrolidone and mixtures thereof.

11. A kit for carrying out the method of claim 1, comprising
i. a single vial with the following components in dried forms
i. a PSMA binding ligand of formula (I): 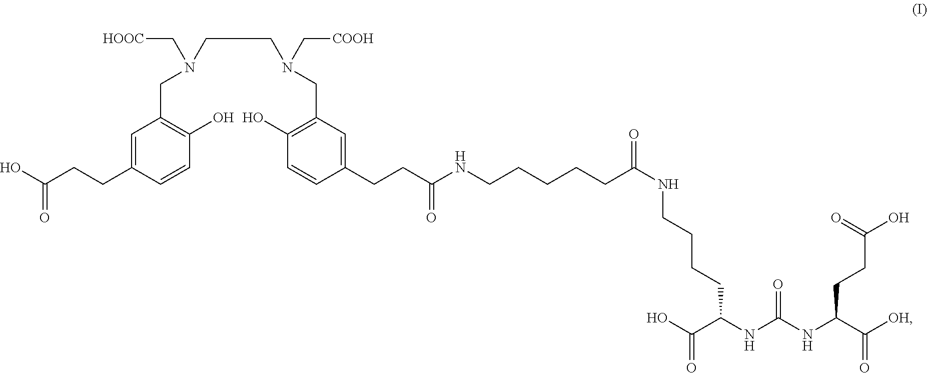 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
and
ii. sodium chloride;
iii. at least one buffering agent, preferably sodium acetate;
iv. a stabilizer against radiolytic degradation, preferably gentisic acid;
v. optionally, an accessory cartridge for eluting a radioactive isotope generated by a radioactive isotope generator or cyclotron.

12. A kit for carrying out the method of claim 1, comprising
i. a single vial with the following components, preferably in dried forms:
i. a PSMA binding ligand of formula (I): 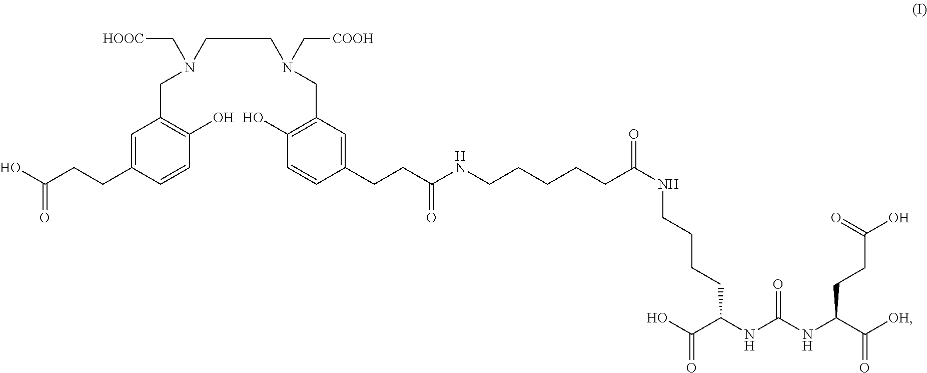 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
and
ii. sodium chloride;
iii. sodium acetate;
iv. a stabilizer against radiolytic degradation, preferably gentisic acid, and;
v. optionally, an accessory cartridge for eluting a radioactive isotope generated by a radioactive isotope generator or cyclotron.

13. The kit of claim 11, wherein said single vial comprises, preferably in dried forms, the following components:
i. a PSMA binding ligand of formula (I) in an amount between 5 μg and 60 μg, preferably between 10 μg and 40 μg, more preferably between 15 μg and 30 μg, even more preferably about 25 μg; 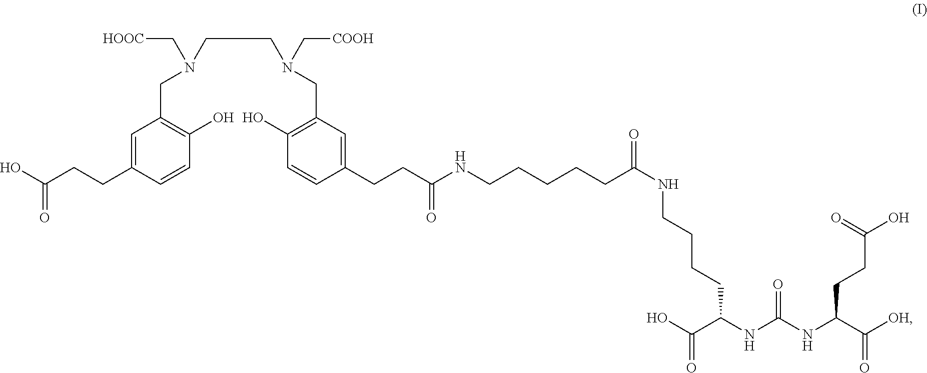 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1]
and
ii. sodium chloride in an amount of at least 10 mg, preferably between 10 mg and 100 mg, more preferably between 30 mg and 50 mg, even more preferably about 40 mg;
iii. sodium acetate in an amount of at least 20 mg, preferably between 20 mg and 80 mg, more preferably between 42 mg and 52 mg, even more preferably about 47 mg;
iv. gentisic acid in an amount of at least 0.2 mg, preferably between 0.5 mg and 2 mg, more preferably between 0.8 mg and 1.2 mg, even more preferably about 1 mg, and;
v. optionally, an accessory cartridge for eluting a radioactive isotope generated by a radioactive isotope generator or cyclotron.

14. The kit of claim 11, wherein said single vial comprises buffering agents suitable for maintaining a pH between 3.0 and 6.0.

15. The kit of claim 11, wherein the kit does not contain any bulking agent selected from the group consisting of carbohydrate and polymeric agent, preferably it does not contain any of the following bulking agents:
mannitol, maltose, trehalose, polyvinylpyrrolidone and mixtures thereof.
